(19) United States
(12) Patent Application Publication
(10) Pub. No.: US 2021/0052771 A1
(43) Pub. Date: Feb. 25, 2021

(54) NANOPARTICULATE MINERALIZED COLLAGEN GLYCOSAMINOGLYCAN SCAFFOLD WITH AN ANTI-RESORPTION FACTOR

(71) Applicants: Brendan A. Harley, Urbana, IL (US); THE REGENTS OF THE UNIVERSITY OF CALIFORNIA, Oakland, CA (US)

(72) Inventors: Brendan A. Harley, Urbana, IL (US); Justine C. Lee, Los Angeles, CA (US); Timothy A. Miller, Los Angeles, CA (US); Xiaoyan Ren, Los Angeles, CA (US)

(73) Assignee: THE REGENTS OF THE UNIVERSITY OF CALIFORNIA, Oakland, CA (US)

(21) Appl. No.: 17/045,470

(22) PCT Filed: Feb. 5, 2019
(86) PCT No.: PCT/US2019/016709

§ 371 Date: Oct. 5, 2020

Related U.S. Application Data
(60) Provisional application No. 62/654,172, filed on Apr. 6, 2018.

(51) Int. Cl. A61L 27/24; A61K 35/28; A61K 35/32; A61K 35/761; A61L 27/38; A61L 27/54; C12N 5/00

(52) U.S. Cl. CPC A61L 27/24; A61K 35/28; A61K 35/32; A61K 35/761; A61L 27/3821; A61L 27/3834; C12N 2533/54; C12N 5/0068; A61L 2300/252; A61L 2300/258; A61L 2300/624; A61L 2300/64; C12N 2533/40; A61L 27/54

(57) ABSTRACT

Compositions including a collagen glycosaminoglycan scaffold and osteoprotegerin are described. The compositions are useful in methods for promoting osteogenesis and attenuating bone resorption.

### CROSS-REFERENCE TO RELATED APPLICATION

This application claims priority under 35 U.S.C. § 119(e) to U.S. Provisional Application 62/654,172 filed Apr. 6, 2018, the entire contents of which are incorporated herein by reference.

### STATEMENT OF GOVERNMENT SUPPORT

This invention was made with government support under the Grant No. IK2 BX002442-01A2 awarded by the Department of Veterans Affairs. Accordingly, the U.S. Government has certain rights to the invention.

### BACKGROUND

Coordination of bone formation and resorption is necessary for the success of bone regenerative strategies. Compositions which can serve as a template for bone growth while limiting bone resorption are needed for the treatment of trauma or congenital deformities affecting bone. The compositions and methods described herein satisfy this need.

### SUMMARY

Provided herein are compositions and methods applicable to bone regenerative strategies. In one aspect, provided is a composition comprising, or alternatively consisting essentially of, or yet further consisting of, a collagen glycosaminoglycan scaffold and one or more of osteoprotegerin (OPG), an OPG fragment or an equivalent of each thereof.

In another aspect, provided are methods of promoting osteogenesis in a subject in need thereof, comprising, or alternatively consisting essentially of, or yet consisting of, administering to the subject an effective amount of a composition, comprising, or alternatively consisting essentially of, or yet consisting of, a collagen glycosaminoglycan scaffold and one or more of osteoprotegerin (OPG), an OPG fragment or an equivalent of each thereof. In another aspect, provided are methods of attenuating bone resorption in a subject in need thereof, comprising or alternatively consisting essentially of, or yet consisting of, administering to the subject an effective amount of a composition comprising or alternatively consisting essentially of, or yet consisting of, a collagen glycosaminoglycan scaffold and one or more of osteoprotegerin (OPG), an OPG fragment or an equivalent of each thereof.

In another aspect, a method of inhibiting osteoclastogenesis in a subject in need thereof is provided, the method comprising, or alternatively consisting essentially of, or yet further consisting of administering to the subject an effective amount of the composition of any embodiment herein.

In another aspect, a method of inhibiting osteoclast activation in a subject in need thereof is provided, comprising, or alternatively consisting essentially of, or yet further consisting of, administering to the subject an effective amount of the composition of any embodiment herein.

In another aspect, provided are methods of preparing a composition, the methods comprising or alternatively consisting essentially of, or yet consisting of, contacting a nanoparticulate non-mineralized collagen glycosaminoglycan (MC-GAG) scaffold with a solution comprising or alternatively consisting essentially of, or yet consisting of one or more of: OPG, an OPG fragment, or an equivalent of each thereof. In another aspect, provided are methods of preparing a composition comprising or alternatively consisting essentially of, or yet consisting of, culturing in a differentiation medium a MSC expressing one or more of exogenous osteoprotegerin (OPG), an exogenous OPG fragment or an equivalent of each thereof; within a collagen glycosaminoglycan scaffold.

In another aspect, provided is a composition prepared by contacting a MC-GAG scaffold with a solution comprising one or more of OPG, an OPG fragment or an equivalent of each thereof. In another aspect, provided is a composition prepared by culturing in a differentiation medium, one or more of an exogenous osteoprotegerin (OPG), an OPG fragment, or an equivalent of each thereof, expressing mesenchymal stem cell (MSC) seeded onto a collagen glycosaminoglycan scaffold.

Yet further provided are kits comprising the compositions as described herein and instructions for use in vitro and/or in vivo.

### BRIEF DESCRIPTION OF THE FIGURES

FIG. 1A: Fluorescent image of AdOPG-transduced primary hMSCs in two-dimensional cultures at 7 days following transduction. FIG. 1B: Western blot of primary hMSCs transduced with control or AdOPG viruses for 7 days on two dimensional cultures.

FIG. 1C: WST-1 proliferation and viability assays of primary hMSCs transduced with control and AdOPG viruses at 3 weeks in 2-dimensional culture.

FIG. 1D: WST-1 proliferation and viability assays of primary hMSCs transduced with control of AdOPG viruses and cultured in osteogenic differentiation medium on non-mineralized collagen glycosaminoglycan (Col-GAG) scaffold or MC-GAG scaffold at 8 weeks. Mean values (n=4) are shown in bars with error bars representing standard deviations.

FIG. 2A: QPCR of control or AdOPG-transduced primary hMSCs cultured on Col-GAG or MC-GAG scaffolds for 14 days in osteogenic differentiation medium for OPG.

FIG. 2B: QPCR of control or AdOPG-transduced primary hMSCs cultured on Col-GAG or MC-GAG scaffolds for 14 days in osteogenic differentiation medium for RANKL.

FIG. 2C: Western blot of control of primary hMSCs cultured on Col-GAG or MC-GAG scaffolds for 56 days in osteogenic differentiation medium for RANKL, OPG, and β-actin in experiment showing AdOPG transduction changes RANKL/OPG homeostasis in primary hMSCs differentiated on Col-GAG and MC-GAG.

FIG. 2D: Western blot of AdOPG-transduced primary hMSCs cultured on Col-GAG or MC-GAG scaffolds for 56 days in osteogenic differentiation medium for RANKL, OPG, and β-actin in experiment showing AdOPG transduction changes RANKL/OPG homeostasis in primary hMSCs differentiated on Col-GAG and MC-GAG.

FIG. 2E: RANKL/OPG gene expression ratio based on QPCR of OPG and RANKL at 14 days of culture.

FIG. 2F: Average RANKL/OPG protein expression ratio based on densitometric analysis of RANKL and OPG western blot bands from 0-56 days. Significant posthoc comparisons following ANOVA indicated with p values.

FIG. 3A: QPCR of control or AdOPG-transduced primary hMSCs cultured on Col-GAG or MC-GAG scaffolds for 14 days in osteogenic differentiation medium for RUNX2.

FIG. 3B: QPCR of control or AdOPG-transduced primary hMSCs cultured on Col-GAG or MC-GAG scaffolds for 14 days in osteogenic differentiation medium for OPN.

FIG. 3C: Western blot of control primary hMSCs cultured on Col-GAG or MC-GAG scaffolds for 56 days in osteogenic differentiation medium for Smad 5 and phosphorylated Smad1/5 (p-Smad1/5).

FIG. 3D: Western blot of AdOPG-transduced primary hMSCs cultured on Col-GAG or MC-GAG scaffolds for 56 days in osteogenic differentiation medium for Smad 5 and phosphorylated Smad1/5 (p-Smad1/5).

FIG. 3E: Representative microCT images of control of AdOPG-transduced primary hMSCs cultured on Col-GAG or MC-GAG scaffolds for 8 weeks. Significant posthoc comparisons following ANOVA indicated with p values.

FIG. 3F: Representative quantitative analysis of control of AdOPG-transduced primary hMSCs cultured on Col-GAG or MC-GAG scaffolds for 8 weeks. Significant posthoc comparisons following ANOVA indicated with p values.

FIG. 4A: Schematic diagram of co-culture design indicating the placement of differentiating hMSCs on Col-GAG or MC-GAG within Transwell insert and lower chamber consisting of primary pre-osteoclasts cultured on a plate coated with calcium phosphate to allow for detection of resorptive pit activity.

FIG. 4B: WST-1 proliferation and viability assays of primary control or AdOPG-transduced hMSCs in single culture (hMSCs Only) or co-cultured (Control hMSC/OC and AdOPG hMSC/OC, respectively) in osteogenic differentiation medium supplemented with RANKL and M-CSF on Col-GAG or MC-GAG for 14 days. Empty, cell-free scaffolds co-cultured with osteoclasts shown for control (Empty Scaffolds/OC).

FIG. 4C: OPG ELISA of hMSC/OC co-culture media (days 3, 7, 10, and 14) with control and AdOPG-transduced hMSCs on Col-GAG and MC-GAG scaffolds. Differentiated osteoclast only (OC Only) culture shown at left as a control.

FIG. 4D: Differentiated osteoclast only (OC Only) culture shown at left as a control. Representative microCT images of empty scaffold (Empty), hMSCs without osteoclasts (hMSC Only), control hMSCs co-cultured with osteoclasts, or AdOPG-transduced hMSCs co-cultured with osteoclasts on Col-GAG or MC-GAG scaffolds for 8 weeks. Significant posthoc comparisons following ANOVA indicated with p values.

FIG. 4E: Differentiated osteoclast only (OC Only) culture shown at left as a control. Representative quantitative analysis of empty scaffold (Empty), hMSCs without osteoclasts (hMSC Only), control hMSCs co-cultured with osteoclasts, or AdOPG-transduced hMSCs co-cultured with osteoclasts on Col-GAG or MC-GAG scaffolds for 8 weeks. Significant posthoc comparisons following ANOVA indicated with p values.

FIG. 5A: WST-1 proliferation and viability assays of primary pre-osteoclasts in single culture (OC Only) or co-cultured with control or AdOPG-transduced hMSCs (Control hMSC/OC and AdOPG hMSC/OC, respectively) in osteogenic differentiation medium supplemented with RANKL and M-CSF on Col-GAG or MC-GAG for 14 days. Empty, cell-free scaffolds co-cultured with osteoclasts shown for control (Empty Scaffold/OC).

FIG. 5B: TRAP staining (upper row), resorption pits (middle row), and live images (lower row) of negative control without cells (No Cells), osteoclast only without hMSCs or scaffolds (OC Only), and osteoclasts co-cultured with Col-GAG or MC-GAG as empty scaffolds (Empty Scaffold), with control hMSCs (Control), or with AdOPG-transduced hMSCs (AdOPG).

FIG. 5C: Quantitative analysis of pit assays as percentage of total area of well in differentiated osteoclasts without hMSCs (OC Only) and osteoclasts co-cultured with Col-GAG and MC-GAG as empty scaffolds, scaffolds with control hMSCs, and scaffolds with AdOPG-transduced hMSCs. Significant posthoc comparisons following ANOVA indicated with p values.

FIGS. 6A and 6B show the results of WST-1 assays of primary pre-osteoclasts. The results of WST-1 assays of primary pre-osteoclasts in single culture (OC Only), co-cultured with empty scaffolds, or co-cultured with control or AdOPG-transduced hMSCs (Control hMSC and AdOPG hMSC, respectively) in medium supplemented with RANKL and M-CSF on Col-GAG or MC-GAG for 14 days (FIG. 6A). Quantitative analysis of pit assays as percentage of total area of well in differentiated osteoclasts without hMSCs (OC Only) and osteoclasts co-cultured with Col-GAG and MC-GAG as empty scaffolds, scaffolds with control hMSCs, and scaffolds with AdOPG-transduced hMSCs. Significant posthoc comparisons following ANOVA indicated with p values (FIG. 6B).

FIGS. 7A-7C: Representative microCT images (FIG. 7A) and quantitative analysis of direct co-cultures of osteoclasts with empty scaffold (Empty+OC), control hMSCs (hMSC+OC), or AdOPG-transduced hMSCs (hMSC/AdOPG+OC) on Col-GAG or MC-GAG for 14 days. Significant posthoc comparisons following ANOVA indicated with p values (FIG. 7B). Western blot of control of AdOPG-transduced primary hMSCs differentiated on Col-GAG or MC-GAG materials co-cultured with osteoclasts for 14 days (FIG. 7C).

FIGS. 8A-8C: illustrates that OPG is expressed and secreted at higher levels by hMSCs on MC-GAG compared to Col-GAG in the absence and presence of differentiating hOCs. Western blot of primary hMSCs cultured on Col-GAG or MC-GAG materials for 0, 3, 14, and 24 days in osteogenic differentiation medium for OPG and β-actin (FIG. 8A). Western blot for p-Smad1/5, total Smad5, p-ERK1/2, ERK1/2, OPG, and β-actin of hMSCs differentiated on Col-GAG or MC-GAG for 3 weeks in the absence and presence of differentiating primary hOCs (FIG. 8B). OPG ELISA of hOCs only, hOCs co-cultured with empty Col-GAG (Empty Col-GAG+hOCs), empty MC-GAG (Empty MC-GAG+hOCs), hMSCs differentiated on Col-GAG (Col-GAG+hMSCs/hOCs), or hMSCs differentiated on MC-GAG (MC-GAG+hMSCs/hOCs) for 4, 7, 11, and 14 days (FIG. 8C). Bars represent mean concentrations in μg/mL, errors bars represent standard deviation. Significant posthoc comparisons following ANOVA indicated with p values.

FIGS. 9A-9B: hMSC mineralization on Col-GAG and MC-GAG is increased in the presence of differentiating hOCs. Representative micro-CT images (FIG. 9A) and quantitative analysis of empty scaffolds, scaffolds cultured with hMSCs only, or scaffolds cultured with hMSCs in co-culture with hOC (hMSC/OC) for 3 weeks (FIG. 9B). Significant posthoc comparisons following ANOVA indicated with p values.

FIGS. 10A-10C: Empty MC-GAG and MC-GAG with differentiating hMSCs diminish the viability, proliferation, and resorption of hOCs. WST-1 proliferation and viability assays of primary hOCs in single culture (OC Only), co-cultured with empty Col-GAG or MC-GAG (Empty Scaffold), or co-cultured with Col-GAG or MC-GAG loaded with hMSCs (Scaffold/hMSCs) after 14 days (FIG. 10A). TRAP staining (upper row), resorption pits (middle row), and live images (lower row) of hOCs in single culture (OC Only) or co-cultured with empty or hMSCs-loaded Col-GAG or MC-GAG (FIG. 10B). Quantitative analysis of pit assays as percentage of total area of well in differentiated osteoclasts without hMSCs (OC Only) and osteoclasts co-cultured with Col-GAG and MC-GAG as empty scaffolds (Empty Scaffold) or scaffolds loaded with differentiating hMSCs (Scaffold/hMSCs) (FIG. 10C). Significant posthoc comparisons following ANOVA indicated with p values.

FIGS. 11A-11B: Western blot of intracellular signaling molecules expressed by hMSCs cultured on Col-GAG and MC-GAG in the absence and presence of DMH1 or PD98059. Western blot of phosphorylated Smad1/5 (P-Smad1/5) and total Smad (Smad5), phosphorylated ERK1/2 (P-ERK1/2) and total ERK1/2 (ERK1/2), OPG, RANKL, and (3-actin in hMSCs cultured on Col-GAG and MC-GAG at day 0, 4, and 24 days of culture with and without 50 μM DMH1 (FIG. 11A) or 50 μM PD98059 (FIG. 11B).

FIGS. 12A and 12B: Mechanisms induced by MC-GAG on osteoprogenitors and osteoclast progenitors (FIG. 12A) and Mechanisms induced by Col-GAG on osteoprogenitors and osteoclast progenitors (FIG. 12B). Referring to FIG. 12A, MC-GAG induces osteogenic differentiation of primary hMSCs via an autogenous activation of the canonical BMPR signaling pathway with phosphorylation of Smad1/5/8 (Mechanism 1). MC-GAG directly inhibits viability, proliferation, and resorptive activity of osteoclasts even in the absence of differentiating hMSCs (Mechanism 2A). MC-GAG also upregulates OPG expression through an ERK1/2 dependent pathway, correlating to an indirect inhibition of resorptive activity but not viability or proliferation in co-culture with differentiating hMSCs (Mechanism 2B). Referring to FIG. 12B, Col-GAG induces osteogenic differentiation of primary hMSCs via both an autogenous activation of the canonical BMPR signaling pathway with phosphorylation of Smad1/5/8 and phosphorylation of ERK1/2 (Mechanism 1). Col-GAG directly inhibits resorptive activity of osteoclasts even in the absence of differentiating hMSCs (Mechanism 2). Although OPG expression is present in hMSCs differentiated on Col-GAG, a net activation of osteoclast mediated resorption occurs through unclear mechanisms (Mechanism 3).

FIG. 13: Sections of 14 mm rabbit calvarial defects reconstructed with Col-GAG or MC-GAG 12 weeks after implantation stained with anti-TRAP and Dapi.

FIGS. 14A-14C: Endogenous OPG secretion from primary hMSCs differentiated on Col-GAG or MC-GAG (FIG. 14A). Elution of OPG from cell-free scaffolds (FIG. 14B) and total endogenous and exogenous soluble OPG from CGO (Col-GAG with OPG bound non-covalently), CGOX (Col-GAG with OPG bound covalently), MCGO (MC-GAG with OPG bound non-covalently), and MCGOX (MC-GAG with OPG bound covalently) (FIG. 14C).

FIG. 15: Regenerated bone defects facilitated by scaffold only versus scaffold+OPG (non-covalent, CGO and MCGO) is different with improved quantity of bone in +OPG scaffolds. The covalently bound versions of these OPG scaffolds are expected to allow for lower and slower release.

### DETAILED DESCRIPTION

Throughout this disclosure, various publications, patents and published patent specifications are referenced by an identifying citation or by an Arabic numeral, the full citation of which is found preceding the claims. The disclosures of these publications, patents and published patent specifications are hereby incorporated by reference into the present disclosure in their entirety to more fully describe the state of the art to which this invention pertains.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

It is to be understood that the terms “subject” and “patient” are interchangeable.

An animal, subject or patient for diagnosis or treatment refers to an animal such as a mammal, or a human, ovine, bovine, feline, canine, equine, simian, etc. Non-human animals subject to diagnosis or treatment include, for example, simians, murine, such as, rat, mice, canine, leporid, livestock, sport animals, and pets. In one aspect, the subject is a human. “Scaffold” as used herein, intends a three dimensional analog of the extracellular matrix.

“Glycosaminoglycan” as used herein, intends a polysaccharide comprising a repeating disaccharide unit which comprises an amino sugar and an uronic sugar.

“Collagen” as used herein, intends the main structural protein of the extracellular space in the connective tissues of animal bodies comprising amino acids wound together to form triple-helices to form elongated fibrils. “Type I collagen” as used herein, intends a type of collagen that forms large eosinophilic fibers known in the art as collagen fibers.

“Mesenchymal stem cell” as used herein, intends multipotent stromal cell that can differentiate into a variety of cell types.

“Osteoprotegerin” or “OPG” as used herein, refers to the glycoprotein also known as osteoclastogenesis inhibitory factor or tumor necrosis factor receptor superfamily member 11B (TNFRSF11B).

“Differentiated,” “Differentiate” and the like, as used herein, refers to the process whereby a cell changes from one cell type to another or changes from one cell type to a more specialized cell type. Non-limiting examples include differentiation of a mesenchymal stem cell to an osteoblast, osteocyte, or osteoclast.

“Osteoblast” as used herein, refers to a cell which is the major cellular component of bone with a single nucleus that synthesizes bone. Osteoblasts are specialized, terminally differentiated products of mesenchymal stem cells. Osteoblasts synthesize dense, cross-linked collagen, osteocalcin, and osteopontin. Osteoblasts mineralize the majority of the bone matrix in air breathing vertebrates.

As used herein, “osteocyte” refers to an osteoblast which is buried within the bone matrix. Osteocytes are the most commonly found cell in mature bone tissue. Osteocytes have a stellate shape, approximately 7 micrometers deep and wide by 15 micrometers in length. The cell body varies in size from 5-20 micrometers in diameter and contains 40-60 cell processes per cell, with a cell to cell distance between 20-30 micrometers. A mature osteocyte contains a single nucleus that is located toward the vascular side and has one or two nucleoli and a membrane. The cell also exhibits a reduced size endoplasmic reticulum, Golgi apparatus and mitochondria, and cell processes that radiate towards the mineralizing matrix. Osteocytes form an extensive connecting syncytial network via small cytoplasmic/dendritic processes in canaliculi.

As used herein, “osteoclast” refers to a type of bone cell that breaks down bone tissue and promotes “bone resorption” as described herein.

“Osteoclastogenesis” refers to a biological process, in which osteoclasts are generated from stem cells.

“Osteoclast activation,” as used herein, refers to the processes which activate osteoclasts to result in bone resorption. Osteoclasts rely on a number of co-factors and process for their activation. Non-limiting examples include the binding Collagen I, the major species in bone extra-cellular matrix (ECM) and the basis for the majority of bone ECM-based regenerative materials, to its ligand for the osteoclast-associated receptor (OSCAR), a co-receptor necessary for osteoclast activation; Fibronectin release; OPG release; and phosphate concentration.

Polynucleotides can have any three dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double and single stranded molecules. Unless otherwise specified or required, any aspect of this technology that is a polynucleotide encompasses both the double stranded form and each of two complementary single stranded forms known or predicted to make up the double stranded form.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “protein,” “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “autologous,” in reference to cells refers to cells that are isolated and infused back into the same subject (recipient or host). “Allogeneic” refers to non-autologous cells.

The term “encode” as it is applied to nucleic acid sequences refers to a polynucleotide which is said to “encode” a polypeptide if, in its native state or when manipulated by methods well known to those skilled in the art, can be transcribed to produce the mRNA for the polypeptide and/or a fragment thereof. Encode also refers to a polynucleotide which is said to “encode” a polypeptide if, in its native state or when manipulated by methods well known to those skilled in the art, can be translated to produce the polypeptide and/or a fragment thereof. The antisense strand is the complement of such a nucleic acid, and the encoding sequence can be deduced therefrom.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality similar to the reference protein, antibody, polypeptide or nucleic acid. Unless specifically recited herein, it is contemplated that any polynucleotide, polypeptide or protein mentioned herein also includes equivalents thereof. For example, an equivalent intends at least about 70% homology or sequence identity, or at least 80% homology or sequence identity and alternatively, or at least about 85%, or alternatively at least about 90%, or alternatively at least about 95%, or alternatively 98% percent homology or sequence identity and exhibits substantially equivalent biological activity to the reference protein, antibody, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

As used herein, “homology” or “identical”, percent “identity” “sequence identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. The terms “homology” or “identical”, percent “identity” “sequence identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

“Exogenous,” “exogenously” and the like are intended to describe a material that is present and active in an organism or cell but that originated outside that organism or cell.

“Endogenous,” “endogenously” and the like, as used herein, describes a protein that originates from the present cell or organism.

As used herein in reference to a polynucleotide, the term “operatively linked” refers to an association between the polynucleotide and the polynucleotide sequence to which it is linked such that, when a specific protein binds to the polynucleotide, the linked polynucleotide is transcribed.

As used herein, the term “fragment,” when referring to a nucleic acid is a nucleic acid having a nucleic acid sequence that is the same as part but not all of the nucleic acid sequence to which “fragment” refers to.

As used herein, the term “overexpress” or “overexpresses” and the like, with respect to a cell, a tissue, or an organ expresses a protein to an amount that is greater than the amount that is produced in a control cell, a control issue, or an organ. A protein that is overexpressed may be endogenous to the host cell or exogenous to the host cell. In one aspect, the protein or polypeptide is expressed at least about 0.25×, or about 0.5×, or about 0.75×, or about 1.0×, or about 1.25×, or about 1.5×, or about 1.75×, or about 2.0×, or more as compared to the normal level in the cell or tissue in its native environment.

“Expression,” “expressing,” “expresses” and the like as used herein, refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

“Recombinant” as used herein, refers to a polypeptide or protein which is produced by recombinant DNA techniques, wherein generally, DNA encoding the polypeptide is inserted into a suitable expression vector which is in turn used to transform a host cell to produce the heterologous protein.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “transduce” or “transduction” and the like, refers to the process whereby a foreign nucleotide sequence is introduced into a cell. In some embodiments, this transduction is done via a vector.

“Nanoparticulate mineralized collagen glycosaminoglycan (MC-GAG) scaffold” as used herein, intends a scaffold that is a substrate for bone regrowth. The scaffold may be used to repair cranial defects and trauma by placement at the site of defect or injury and stimulating bone regeneration at the site. MC-GAG scaffolds may be prepared as known in the art [25-30] or prepared according to methods disclosed herein. “Collagen glycosaminoglycan scaffold” as used herein refers to scaffolds including, but not limited to a MC-GAG scaffold or a Col-GAG scaffold. Collagen glycosaminoglycan scaffolds can be prepared using a lyophilization process of collagen and glycosaminoglycans (GAGs) or collagen-glycosaminoglycan-calcium phosphate produced by combining microfibrillar, type I collagen (Collagen Matrix, Oakland, N.J.) and chondroitin-6-sulfate (Sigma-Aldrich, St. Louis, Mo.) in a solution of 0.005 M to 0.1 M acetic acid, preferably 0.05 M acetic acid (pH 3.2) or with calcium salts not limited to calcium nitrate hydrate: Ca(NO₃)₂.4H₂O or calcium hydroxide: Ca(OH)₂, Sigma-Aldrich) in a solution of phosphoric acid.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

“Administration,” “administering” intends local or systemic administration. In one aspect, local administration is surgical implantation of the compositions described herein. Administration may be accomplished implanting the composition directly or coating or impregnating a surgical implant or prosthesis with the compositions of the disclosure. The compositions may be implanted anywhere throughout the body of the subject where the growth or regeneration of bone is needed. Non-limiting examples include the skull, the facial bones or other bones, large or small in the subject.

Administration or treatment in “combination” refers to administering two agents such that their pharmacological and/or therapeutic effects are manifest at the same time. Combination does not require administration at the same time or substantially the same time, although combination can include such administrations.

An “effective amount” is an amount sufficient to effect beneficial or desired results. An effective amount can be administered in one or more administrations or applications. Such delivery is dependent on a number of variables including the time period for which the individual composition is to be used, the bioavailability of the therapeutic agents included with the composition, the route of administration, etc. It is understood, however, that specific dose levels of the additional therapeutic agents disclosed herein for any particular subject depends upon a variety of factors including the activity of the specific compound employed, bioavailability of the compound, the route of administration, the age of the animal/subject and its body weight, general health, sex, the diet of the animal/subject, the time of administration, the rate of excretion, the drug combination, and the severity of the particular disorder being treated and form of administration. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response such as immunosuppression, osteogenesis, bone resorption or mineralization.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease or trauma), stabilized (i.e., not worsening) state of a condition (including disease or trauma), delay or slowing of condition (including disease or trauma), progression, amelioration or palliation of the condition (including disease or trauma), states and remission (whether partial or total), whether detectable or undetectable. In one aspect, the term “treatment” excludes prevention or prophylaxis.

As used herein, the term “vector” refers to a nucleic acid construct designed for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, adeno-associated viruses (AAVs), etc. according to techniques known in the art. In one embodiment, the viral vector is a lentiviral vector.

As used herein the term “eukaryotic” refers to that which is derived from any organism whose cells have a nucleus enclosed within the membranes. As used herein the term “prokaryotic” refers to that which is derived from a unicellular organism that lacks a membrane bound nucleus, mitochondria or any other membrane bound organelle.

As used herein, “adenoviral” or “adenovirus” refers to medium sized (90-100 nm), non-enveloped (lacking outer lipid bilayer) viruses with an icosahedral nucleocapsid comprising a double-stranded DNA genome. Adenoviruses possess linear double-stranded DNA genome and are able to replicate in the nucleus of vertebrate cells using that cell's replication machinery.

As used herein, “adeno-associated virus,” “adeno-associated viral” or “AAV” refers to a small virus (˜20 nm) that is replication defective and non-enveloped. AAV belongs to the genus Dependoparvovirus and has a genome made of single-stranded DNA, which may be positive or negative-sensed.

As used herein, “alphavirus,” “alphaviral” and the like, refers to a virus which belongs to the group IV Togaviridae family of viruses and has a positive sense, single-stranded RNA genome. Alphaviruses are enveloped with a ˜70 nm diameter with a ˜40 nm nucleocapsid.

As used herein, “lentivirus,” “lentiviral” and the like refers to retroviruses that can integrate their genome into the host's germline genome. “Lentivirus” refers to an enveloped virus, that is slightly pleomorphic measuring ˜80-100 nm in diameter that has two regulatory genes, tat and rev. The nucleocapsids are isometric and the nucleoids are concentric and rod-shaped or truncated-cone shaped.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

A preservative or cryoprotectant can be combined or admixed with the cells, scaffolds, nucleic acids and proteins or compositions containing them. These compositions can be lyophilized using methods known in the art and/or formulated into appropriate dosage forms for ease of use. As used herein, “cryoprotectant” intends a substance used to protect biological tissue from freezing damage. Non-limiting examples include sugars, glycols, dimethyl sulfoxide, and trehalose.

As used herein, “osteogenesis” intends formation of bone, and is meant to include both natural and artificial means of bone formation. As used herein, “bone resorption” or “resorption” intends the process by which osteoclasts break down the tissue in bones and release minerals.

As used herein, “osteoclast” is a type of multinucleated bone cell that breaks down bone tissue through the process of “bone resorption.” Osteoclasts are understood herein to be subject to regulation by receptor activator of nuclear factor κ B (RANK), receptor activator of nuclear factor κ B ligand (RANKL), and OPG.

As used herein, “receptor activator of nuclear factor κ B ligand” or “RANKL” refers to the protein also known as tumor necrosis factor ligand superfamily member 11 (TNFSF11), TNF-related activation-induced cytokine (TRANCE), osteoprotegerin ligand (OPGL), and osteoclast differentiation factor (ODF). In humans, this protein is encoded by the TNFSF 11 gene. RANKL is known as a type II membrane protein and is a member of the tumor necrosis factor (TNF) superfamily. RANKL has been identified to affect the immune system and control bone regeneration and remodeling. RANKL is an apoptosis regulator gene, a binding partner of osteoprotegerin (OPG), a ligand for the receptor RANK and controls cell proliferation by modifying protein levels of Id4, Id2 and cyclin Dl. RANKL is expressed in several tissues and organs including: skeletal muscle, thymus, liver, colon, small intestine, adrenal gland, osteoblast, mammary gland epithelial cells, prostate and pancreas.

As used herein, Receptor Activator of Nuclear Factor κ B (RANK), also known as TRANCE Receptor or TNFRSF11A, is a member of the tumor necrosis factor receptor (TNFR) molecular sub-family. RANK is the receptor for RANK-Ligand (RANKL) and part of the RANK/RANKL/OPG signaling pathway that

regulates osteoclast differentiation and activation. It is associated with bone remodeling and repair, immune cell function, lymph node development, thermal regulation, and mammary gland development. Osteoprotegerin (OPG) is a decoy receptor for RANK, and regulates the stimulation of the RANK signaling pathway by competing for RANKL. The cytoplasmic domain of RANK binds TRAFs 1, 2, 3, 5, and 6 which transmit signals to downstream targets such as NF-κB and JNK. RANK is constitutively expressed in skeletal muscle, thymus, liver, colon, small intestine, adrenal gland, osteoclast, mammary gland epithelial cells, prostate, vascular cell, and pancreas. Most commonly, activation of NF-κB is mediated by RANKL, but over-expression of RANK alone is sufficient to activate the NF-κB pathway. RANK is a 616 amino acid type I transmembrane protein. Its extracellular domain consists of 184 amino acids, its transmembrane domain has 21 amino acids, and its cytoplasmic domain consists of 383 amino acids. Like other members of the TNFR family, it has four extracellular cysteine-rich pseudo-repeat domains (CRDs). It shares 40% amino acid identity with CD40. RANK is encoded on human chromosome 18q22.1. It shows 85% homology between mouse and human homologues. RANKL binds to RANK, which then binds to TRAF6. TRAF6 stimulates the activation of the c-jun N-terminal kinase (JNK) and nuclear factor kappa-b (NF-kB) pathways which trigger differentiation and activation of osteoclasts. This system is balanced by the relative expression of OPG to RANKL, which are highly regulated by many factors including hormones, immune signals, and growth factors. An overexpression of RANKL can cause an overproduction and activation of osteoclasts, which break down bone.

As used herein, “iliac crest” of a subject intends the superior border of the wing of ilium and the superolateral margin of the greater pelvis. The iliac crest has a large amount of red bone marrow, and thus it is the site of bone marrow harvests to collect stem cells.

As used herein, “harvest” intends removal of biological material from the subject. A non-limiting example of harvesting biological material is harvesting stem cells. Stem cells may be harvested from a subject for either autologous or allogenic use in the same or different subject. Harvest of stem cells can be accomplished by methods known to the skilled artisan. Non-limiting examples of doing so include harvest from bone marrow or harvest from peripheral blood.

As used herein, “seeding” intends incorporation or infusion of MSCs into and/or onto a collagen glycosaminoglycan scaffold (MC-GAG or Col-GAG scaffold). Seeding can be accomplished using techniques known to the skilled artisan, including, but not limited to placement of a suspension of mesenchymal stem cells in growth media and pipetting this mixture onto the scaffold.

As used herein, “medium” refers to a growth medium or culture medium that is a solid, liquid or semi-solid designed to support the growth of cells. “Differentiation medium” refers to a medium specifically for inducing differentiating of an MSC. Non-limiting examples of components of a differentiation medium for MSCs include, fetal-bovine serum, penicillin-streptomycin, glutamine, β-glycerophosphate, ascorbic acid, and dexamethasone.

### Modes for Carrying Out the Disclosure

Materials inspired by bone-specific extracellular matrix (ECM) components, such as the nanoparticulate mineralized collagen glycosaminoglycan scaffold have generated great enthusiasm in regenerative technologies due to their abilities to instruct osteoprogenitor differentiation. Osteoclasts present the potential for modulation of resorption within the host microenvironment via alterations of the receptor activator of nuclear factor-κB (RANK), RANK ligand (RANKL), and osteoprotegerin (OPG) axis [1-5].

The RANK/RANKL/OPG axis serves an important role in osteoclast regulation and bone homeostasis [6-7]. RANK, a tumor necrosis factor superfamily receptor originally identified in T lymphocytes and osteoblasts, via its cognate ligand RANKL, is required for osteoclast differentiation and activation [8, 9]. Murine genetic models have shown that both RANK and RANKL deficiencies result in osteopetrosis due to a complete absence of osteoclasts [10, 11]. In the craniofacial skeleton, RNA interference using small interfering RNAs (siRNA) specific for RANK has been shown to fuse patent cranial sutures in ex vivo cultures [6]. OPG, the soluble decoy receptor for RANKL, is the major, endogenous negative regulator of the pathway. In contrast to the RANK and RANKL-deficient mice, OPG knockouts exhibit profound osteoporosis [11, 12]. Due to the direct relationship between the RANK/RANKL/OPG axis to osteoclast activation, this axis presents a target for therapies directed to fracture healing and other conditions requiring a net osteogenic state [13].

The importance of osteoclast homeostasis in normal bone physiology suggests that bone regeneration is likely to be affected by the regulatory mechanisms for osteoclast activation. Components of extracellular matrix (ECM)-based materials have been reported to effect both osteogenic differentiation and negatively or positively regulate osteoclastogenesis [16]. As the most abundant protein within bone ECM, most ECM-inspired materials for bone regeneration are based on collagen I. However, the ligands for OSCAR, a co-stimulatory molecule for osteoclast maturation, are collagen I, II, and III [14]. Thus, collagen-based materials intrinsically provide co-stimulation for osteoclast activation, potentially lowering the threshold for resorption. Collagen-based osteoclast costimulation is likely able to be offset with the negative osteoclast-regulatory effects of certain glycosaminoglycan (GAG) species as well as the inorganic components of bone ECM.

Nanoparticulate mineralized collagen glycosaminoglycan material (MC-GAG) induces efficient mineralization of bone marrow-derived primary human mesenchymal stem cells (hMSCs) and primary rabbit bone marrow stromal cells (rBMSCs) in a manner that required an autogenous activation of the bone morphogenetic protein receptor (BMPR) signaling pathway through phosphorylation of small mothers against decapentaplegic-1/5 (Smad1/5) [25-30]. Furthermore, MC-GAG induces in vivo rabbit calvarial regeneration without the addition of exogenous growth factors or progenitor cells [28].

In osteoclast regulation, MC-GAG demonstrated both direct and indirect inhibitory effects on osteoclast viability, proliferation, and activation. In comparison to its non-mineralized collagen glycosaminoglycan (Col-GAG) counterpart, MC-GAG also induces hMSCs to express higher levels of OPG early in osterogenic differentiation via intracellular signaling pathways distinct from those governing osteogenic differentiation.

Disclosed herein are compositions and methods with increased OPG levels relative to what is naturally present in the organism or possible with implantation of MC-GAG alone. Thus, osteoclasts are regulated via the RANK/RANKL/OPG axis using collagen glycosaminoglycan scaffolds to facilitate bone regeneration.

Compositions

In one aspect, provided are compositions comprising, or consisting essentially of, or yet further consisting or, one or more of a collagen glycosaminoglycan scaffold and osteoprotegerin (OPG), an OPG fragment or an equivalent of each thereof. As used herein and unless specifically noted otherwise, OPG intends full length or a fragment of the protein, as well as mammalian OPG and biological equivalents thereof. In a further aspect, the compositions are combined with a carrier, such as a pharmaceutically acceptable carrier, and optionally a cryoprotectant or preservative. The compositions can be formulated and lyophilized or frozen for ease of storage and use. In addition, they can be provided in specific dosages for ease of administration.

As used herein, a fragment of OPG or OPG fragment intends the minimal amino acid sequence that is necessary to bind to its receptor. The OPG can be isolated or purified from a suitable source, such as a mammal or is recombinantly produced. In some embodiments, the OPG is expressed from a nucleic acid that comprises, or consists essentially of, or yet further consists of, the polynucleotide of SEQ ID NO: 1 or a homolog thereof, a biological equivalent thereof, or a nucleic acid having at least 80%, or alternatively at least 85%, or at least 90%, or at least 95%, or at least 98% sequence identity to SEQ ID NO: 1, or a fragment of each thereof that encodes functional OPG or a fragment thereof. In some embodiments, the OPG is expressed from a nucleic acid that comprises, or consists essentially of, or yet further consists of, a polynucleotide encoding SEQ ID NO: 2 or a homolog thereof, a biological equivalent thereof, or a nucleic acid having at least 80%, or alternatively at least 85%, or at least 90%, or at least 95%, or at least 98% sequence identity to a polynucleotide encoding SEQ ID NO: 2, or a fragment of each thereof that encodes functional OPG or a fragment thereof. In some embodiments, the OPG has the amino acid sequence of SEQ ID NO: 2, or a homolog thereof, a biological equivalent thereof, or a polypeptide having at least 80%, or alternatively at least 85%, or at least 90%, or at least 95%, or at least 98% sequence identity to SEQ ID NO: 2, or a fragment of each thereof that is functional OPG or a fragment thereof. In some embodiments, the OPG is acquired commercially from sources not limited to Peprotech, LifeSpan Biosciences, Zageno, and ThermoFisher. The compositions are prepared according to methods described herein or known in the art.

In some embodiments, the collagen glycosaminoglycan scaffold is a nanoparticulate mineralized collagen glycosaminoglycan (MC-GAG) scaffold. In some embodiments, the collagen glycosaminoglycan scaffold is a non-mineralized collagen glycosaminoglycan (Col-GAG) scaffold. In some embodiments, the collagen is type I collagen. In some embodiments, the collagen glycosaminoglycan scaffold comprises a porosity of about 10%, 15% 20%, 25%, 30%, 35%, 40%, 45%, 50%, 55%, 60%, 65%, 70%, 75%, 80%, 85%, 90%, or 95%. In some embodiments the collagen glycosaminoglycan scaffold comprises a porosity of about 40%. In some embodiments the collagen glycosaminoglycan scaffold comprises a porosity of about 45%. In some embodiments the collagen glycosaminoglycan scaffold comprises a porosity of about 50%. In some embodiments the collagen glycosaminoglycan scaffold comprises a porosity of about 55%. In some embodiments the collagen glycosaminoglycan scaffold comprises a porosity of about 60%. In some embodiments the collagen glycosaminoglycan scaffold comprises a porosity of about 65%. In some embodiments the collagen glycosaminoglycan scaffold comprises a porosity of about 70%. In some embodiments the collagen glycosaminoglycan scaffold comprises a porosity of about 75%. In some embodiments the collagen glycosaminoglycan scaffold comprises a porosity of about 80%. In some embodiments the collagen glycosaminoglycan scaffold comprises a porosity of about 85%. In some embodiments the collagen glycosaminoglycan scaffold comprises a porosity of about 90%. In some embodiments the collagen glycosaminoglycan scaffold comprises a porosity of about 95%.

In some embodiments the collagen glycosaminoglycan scaffold comprises a pore size between about 5 μm to about 10 μm. In some embodiments the collagen glycosaminoglycan scaffold comprises a pore size between about 10 μm to about 40 μm. In some embodiments the collagen glycosaminoglycan scaffold comprises a pore size between about 40 μm to about 70 μm. In some embodiments the collagen glycosaminoglycan scaffold comprises a pore size between about 70 μm to about 100 μm. In some embodiments the collagen glycosaminoglycan scaffold comprises a pore size between about 100 μm to about 130 μm. In some embodiments the collagen glycosaminoglycan scaffold comprises a pore size between about 130 μm to about 160 μm. In some embodiments the collagen glycosaminoglycan scaffold comprises a pore size between about 160 μm to about 190 μm. In some embodiments the collagen glycosaminoglycan scaffold comprises a pore size between about 210 μm to about 240 μm. In some embodiments the collagen glycosaminoglycan scaffold comprises a pore size greater than 240 μm.

In some embodiments the morphology of the scaffold comprises isotropic pores with a transverse: longitudinal pore aspect ratio of about 0.05. In some embodiments the morphology of the scaffold comprises isotropic pores with a transverse: longitudinal pore aspect ratio of about 0.15. In some embodiments collagen glycosaminoglycan scaffold comprises isotropic pores with a transverse: longitudinal pore aspect ratio of about 0.25. In some embodiments the morphology of the scaffold comprises isotropic pores with a transverse: longitudinal pore aspect ratio of about 0.35. In some embodiments collagen glycosaminoglycan scaffold comprises isotropic pores with a transverse: longitudinal pore aspect ratio of about 0.45. In some embodiments the collagen glycosaminoglycan scaffold comprises isotropic pores with a transverse: longitudinal pore aspect ratio of about 0.55. In some embodiments the collagen glycosaminoglycan scaffold comprises isotropic pores with a transverse: longitudinal pore aspect ratio of about 0.65. In some embodiments the collagen glycosaminoglycan scaffold comprises isotropic pores with a transverse: longitudinal pore aspect ratio of about 0.75. In some embodiments the collagen glycosaminoglycan scaffold comprises isotropic pores with a transverse: longitudinal pore aspect ratio of about 0.85. In some embodiments the collagen glycosaminoglycan scaffold comprises isotropic pores with a transverse: longitudinal pore aspect ratio of about 0.95. In some embodiments the collagen glycosaminoglycan scaffold comprises isotropic pores with a transverse: longitudinal pore aspect ratio of about 0.99.

In some embodiments, the OPG, OPG fragment or equivalent of each thereof is provided by a mesenchymal stem cell (MSC) or a cell differentiated from a MSC, that expresses the (OPG), the OPG fragment or the equivalent of each thereof. In some embodiments, the OPG, OPG fragment or equivalent of each thereof is expressed at a level above endogenously expressed OPG. In some embodiments, the OPG, OPG fragment or equivalent of each thereof is expressed at a level above about 2 ng/mL. In some embodiments, the OPG, OPG fragment or equivalent of each thereof is expressed at a level above about 3 ng/mL. In some embodiments, the OPG, OPG fragment or equivalent of each thereof is expressed at about 5 ng/mL to about 20 ng/mL. In some embodiments, the OPG, OPG fragment or equivalent of each thereof is recombinant.

Levels of OPG, OPG fragment or equivalent of each thereof expressed by MSC can be determined by analysis of a sample of the microenvironment surrounding the MSC using enzyme linked immunosorbent assay, gas chromatography mass spectrometry, 2-dimensional electrophoresis, spectrophotometric techniques, matrix-assisted laser desorption/ionization (MALDI) ionization mass spectrometry, or time of flight (TOF) mass spectrometry.

In some embodiments, the OPG, the OPG fragment or the equivalent of each thereof is encoded by a nucleic acid, wherein the nucleic acid comprises (i) a polynucleotide of SEQ ID NO: 1; (ii) a polynucleotide comprising a biological equivalent of SEQ ID NO: 1; (iii) a polynucleotide having at least 80%, or alternatively at least 85%, or at least 90%, or at least 95%, or at least 98% sequence identity to SEQ ID NO: 1; or (iv) a fragment of the polynucleotide of any one of (i)-(iii) that encodes functional OPG. In some embodiments, the OPG is expressed from a nucleic acid that comprises, or consists essentially of, or yet further consists of, a polynucleotide encoding SEQ ID NO: 2 or a homolog thereof, a biological equivalent thereof, or a nucleic acid having at least 80%, or alternatively at least 85%, or at least 90%, or at least 95%, or at least 98% sequence identity to a polynucleotide encoding SEQ ID NO: 2, or a fragment of each thereof that encodes functional OPG or a fragment thereof. In some embodiments, the OPG has the amino acid sequence of SEQ ID NO: 2, or a homolog thereof, a biological equivalent thereof, or a polypeptide having at least 80%, or alternatively at least 85%, or at least 90%, or at least 95%, or at least 98% sequence identity to SEQ ID NO: 2, or a fragment of each thereof that is functional OPG or a fragment thereof.

Compositions comprising the compounds described herein can be manufactured by means of conventional mixing, dissolving, granulating, dragee-making levigating, emulsifying, encapsulating, entrapping, or lyophlization processes. The compositions can be formulated in conventional manner using one or more physiologically acceptable carriers, diluents, excipients, or auxiliaries which facilitate processing of the compounds provided herein into preparations which can be used in vitro or in vivo. In some embodiments, the nucleic acid encoding OPG, OPG fragment or equivalent of each thereof is operatively linked to one or more regulatory elements that provide for the expression of the nucleic acid, optionally the nucleic acid and the one or more regulatory elements are comprised within a vector. In some embodiments, the vector is selected from a eukaryotic vector or a prokaryotic vector. In some embodiments, the eukaryotic vector is selected from the group of an adenoviral vector an alphaviral vector, an adeno-associated viral vector (AAV), and a lentiviral vector. In some embodiments, elements for the expression of the polynucleotide comprise a promoter, the correct translation initiation sequence such as a ribosomal binding site and start codon, a termination codon, or a transcription termination sequence.

In some embodiments, the MSC is a bone marrow derived MSC. In some embodiments, the MSC is an adipose tissue derived MSC. In some embodiments, the MSC is a peripheral blood derived MSC. In some embodiments, the MSC is a periodontal ligament derived MSC. In some embodiments, the MSC is a dentition derived MSC. In some embodiments, the MSC is a urine derived MSC. In some embodiments, the MSC is a mammalian MSC, non-limiting examples of such include a bovine MSC, a feline MSC, a canine MSC, a murine MSC, an equine MSC and a human MSC. In some embodiments, the MSC is a human MSC. In some embodiments, the human MSC has a cell marker profile comprising CD105⁺, CD166⁺, CD29⁺, CD44⁺, CD14⁻, CD34⁻, and CD45⁻. In some aspects, the MSC is differentiated into a differentiation product, e.g., an osteoblast or an osteocyte.

In some embodiments, the composition further comprises, or consists essentially of, or yet further consist of, a carrier. In some embodiments, the carrier further comprises one or more of a cryoprotectant or a preservative.

In another aspect, provided is a composition prepared by contacting a MC-GAG scaffold with a solution comprising OPG, an OPG fragment or an equivalent of each thereof. In some embodiments, the solution comprises OPG in phosphate buffered saline.

In another aspect, provided is a composition prepared by culturing in a differentiation medium, an exogenous osteoprotegerin (OPG) expressing mesenchymal stem cell (MSC) seeded into a collagen glycosaminoglycan scaffold, to produce the composition. The compositions can be further processed for storage or transport, e.g., by freezing or the like.

In some embodiments the OPG, OPG fragment or equivalent of each thereof is in the solution at a concentration of about 0.5 μg/mL to about 10 μg/mL. In some embodiments the OPG, OPG fragment or equivalent of each thereof is in the solution at a concentration of about 1 μg/mL to about 7 μg/mL. In some embodiments the OPG, OPG fragment or equivalent of each thereof is in the solution at a concentration of about 2 μg/mL to about 6 μg/mL. In some embodiments the OPG, OPG fragment or equivalent of each thereof is in the solution at a concentration of about 3 μg/mL to about 5 μg/mL. In some embodiments the OPG, OPG fragment or equivalent of each thereof is in the solution at a concentration of about 4 μg/mL to about 4.5 μg/mL.

Therapeutic Methods

In another aspect, provided are methods of promoting osteogenesis in a subject in need thereof, the methods comprising, or alternatively consisting essentially of, or yet further consisting of, administering to the subject an effective amount of a composition comprising a collagen glycosaminoglycan scaffold and one or more of osteoprotegerin (OPG), an OPG fragment or an equivalent of each thereof. As used herein and unless specifically noted otherwise, OPG intends full length or a fragment of the protein, as well as mammalian OPG and biological equivalents thereof. Effective amounts can be determined by the treating physician or veterinarian, and will vary with the subject being treated, the composition being used and the indication.

As used herein, a fragment of OPG or OPG fragment intends the minimal amino acid sequence that is necessary to bind to its receptor. The OPG can be isolated or purified from a suitable source, such as a mammal or is recombinantly produced. In some embodiments, the OPG is expressed from a nucleic acid that comprises, or consists essentially of, or yet further consists of, the polynucleotide of SEQ ID NO: 1 or a homolog thereof, a biological equivalent thereof, or a nucleic acid having at least 80%, or alternatively at least 85%, or at least 90%, or at least 95%, or at least 98% sequence identity to SEQ ID NO: 1, or a fragment of each thereof that encodes functional OPG or a fragment thereof. In some embodiments, the OPG is expressed from a nucleic acid that comprises, or consists essentially of, or yet further consists of, a polynucleotide encoding SEQ ID NO: 2 or a homolog thereof, a biological equivalent thereof, or a nucleic acid having at least 80%, or alternatively at least 85%, or at least 90%, or at least 95%, or at least 98% sequence identity to a polynucleotide encoding SEQ ID NO: 2, or a fragment of each thereof that encodes functional OPG or a fragment thereof. In some embodiments, the OPG has the amino acid sequence of SEQ ID NO: 2, or a homolog thereof, a biological equivalent thereof, or a polypeptide having at least 80%, or alternatively at least 85%, or at least 90%, or at least 95%, or at least 98% sequence identity to SEQ ID NO: 2, or a fragment of each thereof that is functional OPG or a fragment thereof. In some embodiments, the OPG is acquired commercially from sources not limited to Peprotech, LifeSpan Biosciences, Zageno, or ThermoFisher.

In another aspect, provided are methods of attenuating bone resorption in a subject in need thereof, the methods comprising, or alternatively consisting essentially of, or yet further consisting of, administering to the subject an effective amount of a composition as described herein. In some embodiments, the subject is a mammal. In some embodiments, the subject is a human.

In some embodiments of the methods, the composition comprises a mesenchymal stem cell (MSC), expressing exogenous osteoprotegerin (OPG), an exogenous OPG fragment or an exogenous equivalent of each thereof wherein the MSC is autologous to the subject. In some embodiments, the MSC autologous to the subject is harvested from the iliac crest, peripheral blood, or femoral epiphysis of the subject. In some embodiments, the MSC autologous to the subject is harvested from the iliac crest of the subject. In some embodiments, the MSC autologous to the subject is harvested from the peripheral blood of the subject. In some embodiments, the MSC autologous to the subject is harvested from the femoral epiphysis of the subject. In some embodiments, the MSC autologous to the subject is harvested from adipose tissue of the subject. In some embodiments, the MSC autologous to the subject is harvested from periodontal ligament tissue of the subject. In some embodiments, the MSC autologous to the subject is harvested from dentition of the subject. In some embodiments, the MSC autologous to the subject is harvested from urine or other bodily fluids of the subject. In some embodiments, the composition is implanted into the subject. In some embodiments, the composition is implanted into the subject surgically. Other modes of administration are within the scope of this disclosure.

In some refinements, the collagen glycosaminoglycan scaffold comprises about 2 μg of OPG, OPG fragment or equivalent of each thereof per 2 cm² of collagen glycosaminoglycan scaffold. In some refinements, the collagen glycosaminoglycan scaffold comprises about 4 μg of OPG, OPG fragment or equivalent of each thereof per 2 cm² of collagen glycosaminoglycan scaffold. In some refinements, the collagen glycosaminoglycan scaffold comprises about 6 μg of OPG, OPG fragment or equivalent of each thereof per 2 cm² of collagen glycosaminoglycan scaffold. In some refinements, the collagen glycosaminoglycan scaffold comprises about 7 μg of OPG, OPG fragment or equivalent of each thereof per 2 cm² of collagen glycosaminoglycan scaffold. In some refinements, the collagen glycosaminoglycan scaffold comprises about 8 μg of OPG, OPG fragment or equivalent of each thereof per 2 cm² of collagen glycosaminoglycan scaffold. In some refinements, the collagen glycosaminoglycan scaffold comprises about 10 μg of OPG, OPG fragment or equivalent of each thereof per 2 cm² of collagen glycosaminoglycan scaffold. In some refinements, the collagen glycosaminoglycan scaffold comprises about 12 μg of OPG, OPG fragment or equivalent of each thereof per 2 cm² of collagen glycosaminoglycan scaffold. In some refinements, the collagen glycosaminoglycan scaffold comprises about 18 μg of OPG, OPG fragment or equivalent of each thereof per 2 cm² of collagen glycosaminoglycan scaffold. In some refinements, the collagen glycosaminoglycan scaffold comprises about 50 μg of OPG, OPG fragment or equivalent of each thereof per 2 cm² of collagen glycosaminoglycan scaffold. As is understood to the skilled artisan, an effective amount of the compositions is administered either locally or systemically. In one aspect the compositions are contacted with a tissue requiring treatment that may be in vivo or in vitro. When practiced in vitro, the method provides an assay to test for combination therapies.

One of skill in the art can determine when the purpose of the methods described herein have been accomplished by various clinical endpoints such as the growth of new bone tissue. The growth of new bone tissue in vivo can be determined through diagnostic techniques including, but not limited to, computed tomography (CT) scan, or magnetic resonance imaging (MRI). In vitro, one of skill in the art can determine when the purpose of the methods described herein have been accomplished using histology.

Methods of Preparation

In one aspect, provided are methods of preparing a composition comprising contacting a MC-GAG scaffold with a solution comprising OPG, an OPG fragment or an equivalent of each thereof. As used herein and unless specifically noted otherwise, OPG intends full length or a fragment of the protein, as well as mammalian OPG and biological equivalents thereof. In one embodiment, the OPG is provided in a carrier such as phosphate buffered saline.

In one embodiment, the composition comprises, or alternatively consists essentially of, or yet further consists of one or more of OPG, a fragment or an equivalent of each thereof non-covalently incorporated therein. In one embodiment the method comprises, consists essentially or, or yet further consists of lyophilizing a suspension comprising, or alternatively consisting essentially of, or yet further consisting of microfibrillar type I collagen and chondroitin-6-sulfate in a solution comprising, or alternatively consisting essentially of, or yet further consisting of acetic acid. In some embodiments, the solution further comprises, or alternatively consists essentially of, or yet further consists of OPG or a fragment thereof. In some embodiments, the solution lyophilized does not comprise, or alternatively consist essentially of, or yet further consist of OPG or a fragment thereof and the process further comprises, or alternatively consists essentially of, or yet further consists of freezing the solution and sublimating the frozen solution to produce a scaffold, contacting the scaffold with a solution comprising, consisting essentially of, or consisting of 1-ethyl-3-(3-dimethylaminopropyl) carbodiimide and N-hydroxysuccinimide, and contacting the scaffold with a solution comprising, or alternatively consisting essentially of, or yet further consisting of OPG or a fragment thereof, to produce the composition.

In one embodiment, the composition comprises, or alternatively consists essentially of, or yet further consists of one or more of OPG, a fragment or an equivalent of each thereof covalently incorporated therein. In one embodiment the method comprises, consists essentially or, or yet further consists of lyophilizing a suspension comprising, or alternatively consisting essentially of, or yet further consisting of microfibrillar type I collagen, chondroitin-6-sulfate and calcium salts in a solution comprising, or alternatively consisting essentially of, or yet further consisting of phosphoric acid. In some embodiments, the calcium salts comprise, or alternatively consist essentially of, or yet further consist of Ca(NO₃)₂ and Ca(OH)₂. In some embodiments, the solution further comprises, or alternatively consists essentially of, or yet further consists of OPG or a fragment thereof. In some embodiments, the solution lyophilized does not comprise, or alternatively consist essentially of, or yet further consist of OPG or a fragment thereof and the process further comprises, or alternatively consists essentially of, or yet further consists of freezing the solution and sublimating the frozen solution to produce a scaffold, contacting the scaffold with a solution comprising, consisting essentially of, or consisting of 1-ethyl-3-(3-dimethylaminopropyl) carbodiimide, N-hydroxysuccinimide, and OPG or a fragment thereof, to produce the composition.

In some embodiments the OPG, OPG fragment or equivalent of each thereof is in the solution at a concentration of about 0.5 μg/mL to about 10 μg/mL. In some embodiments the OPG, OPG fragment or equivalent of each thereof is in the solution at a concentration of about 1 μg/mL to about 7 μg/mL. In some embodiments the OPG, OPG fragment or equivalent of each thereof is in the solution at a concentration of about 2 μg/mL to about 6 μg/mL. In some embodiments the OPG, OPG fragment or equivalent of each thereof is in the solution at a concentration of about 3 μg/mL to about 5 μg/mL. In some embodiments the OPG, OPG fragment or equivalent of each thereof is in the solution at a concentration of about 4 μg/mL to about 4.5 μg/mL.

As used herein, a fragment of OPG or OPG fragment intends the minimal amino acid sequence that is necessary to bind to its receptor. The OPG can be isolated or purified from a suitable source, such as a mammal or is recombinantly produced. In some embodiments, the OPG is expressed from a nucleic acid that comprises, or consists essentially of, or yet further consists of, the polynucleotide of SEQ ID NO: 1 or a homolog thereof, a biological equivalent thereof, or a nucleic acid having at least 80%, or alternatively at least 85%, or at least 90%, or at least 95%, or at least 98% sequence identity to SEQ ID NO: 1, or a fragment of each thereof that encodes functional OPG or a fragment thereof. In some embodiments, the OPG is expressed from a nucleic acid that comprises, or consists essentially of, or yet further consists of, a polynucleotide encoding SEQ ID NO: 2 or a homolog thereof, a biological equivalent thereof, or a nucleic acid having at least 80%, or alternatively at least 85%, or at least 90%, or at least 95%, or at least 98% sequence identity to a polynucleotide encoding SEQ ID NO: 2, or a fragment of each thereof that encodes functional OPG or a fragment thereof.

In another aspect, provided herein is a method of preparing a composition comprising culturing in a differentiation medium, an exogenous osteoprotegerin (OPG) expressing mesenchymal stem cell (MSC) seeded onto a collagen glycosaminoglycan scaffold. In some embodiments, the differentiation medium comprises one or more of a compound selected from the group consisting of β-glycerophosphate, ascorbic acid, and dexamethasone. In some embodiments, the composition comprises a mesenchymal stem cell (MSC), expressing exogenous osteoprotegerin (OPG) or a fragment or an equivalent of each thereof. As used herein and unless specifically noted otherwise, OPG intends full length or a fragment of the protein, as well as mammalian OPG and biological equivalents thereof.

In some embodiments, the MC-GAG scaffold is sterilized. In some embodiments, the MC-GAG scaffold is sterilized with ethylene oxide and crosslinked in a solution of 1-ethyl-3-(3-dimethylaminopropyl) carbodiimide and N-hydroxysuccinimide.

As used herein, a fragment of OPG intends the minimal amino acid sequence that is necessary to bind to its receptor. The OPG can be isolated or purified from a suitable source, such as a mammal or is recombinantly produced. In some embodiments, the OPG is expressed from a nucleic acid that comprises, or consists essentially of, or yet further consists of, the polynucleotide of SEQ ID NO: 1 or a homolog thereof, a biological equivalent thereof, or a nucleic acid having at least 80%, or alternatively at least 85%, or at least 90%, or at least 95%, or at least 98% sequence identity to SEQ ID NO: 1, or a fragment of each thereof that encodes functional OPG or a fragment thereof. In some embodiments, the OPG is expressed from a nucleic acid that comprises, or consists essentially of, or yet further consists of, a polynucleotide encoding SEQ ID NO: 2 or a homolog thereof, a biological equivalent thereof, or a nucleic acid having at least 80%, or alternatively at least 85%, or at least 90%, or at least 95%, or at least 98% sequence identity to a polynucleotide encoding SEQ ID NO: 2, or a fragment of each thereof that encodes functional OPG or a fragment thereof. In some embodiments, the OPG has the amino acid sequence of SEQ ID NO: 2, or a homolog thereof, a biological equivalent thereof, or a polypeptide having at least 80%, or alternatively at least 85%, or at least 90%, or at least 95%, or at least 98% sequence identity to SEQ ID NO: 2, or a fragment of each thereof that is functional OPG or a fragment thereof. In another embodiment of any of the above methods, the composition comprises a mesenchymal stem cell (MSC), expressing exogenous osteoprotegerin (OPG) wherein the MSC has been transduced with a virus comprising a nucleic acid encoding OPG or an OPG fragment or an equivalent of each thereof. As used herein and unless specifically noted otherwise, OPG intends full length or a fragment of the protein, as well as mammalian OPG and biological equivalents thereof.

As used herein, a fragment of OPG or OPG fragment intends the minimal amino acid sequence that is necessary to bind to its receptor. The OPG or OPG fragment can be isolated or purified from a suitable source, such as a mammal or is recombinantly produced. In some embodiments, the OPG or OPG fragment is expressed from a nucleic acid that comprises, or consists essentially of, or yet further consists of, the polynucleotide of SEQ ID NO: 1 or a homolog thereof, a biological equivalent thereof, or a nucleic acid having at least 80%, or alternatively at least 85%, or at least 90%, or at least 95%, or at least 98% sequence identity to SEQ ID NO: 1, or a fragment of each thereof that encodes functional OPG or a functional OPG fragment thereof. In some embodiments, the OPG is expressed from a nucleic acid that comprises, or consists essentially of, or yet further consists of, a polynucleotide encoding SEQ ID NO: 2 or a homolog thereof, a biological equivalent thereof, or a nucleic acid having at least 80%, or alternatively at least 85%, or at least 90%, or at least 95%, or at least 98% sequence identity to a polynucleotide encoding SEQ ID NO: 2, or a fragment of each thereof that encodes functional OPG or a fragment thereof. In some embodiments, the OPG has the amino acid sequence of SEQ ID NO: 2, or a homolog thereof, a biological equivalent thereof, or a polypeptide having at least 80%, or alternatively at least 85%, or at least 90%, or at least 95%, or at least 98% sequence identity to SEQ ID NO: 2, or a fragment of each thereof that is functional OPG or a fragment thereof.

In some embodiments, the nucleic acid encoding OPG comprises the polynucleotide of SEQ ID NO: 1, a biological equivalent thereof, or a nucleic acid having at least 80%, or alternatively at least 85%, or at least 90%, or at least 95%, or at least 98% sequence identity to SEQ ID NO: 1. In some embodiments, the virus is selected from the group consisting of an adenovirus, an alphavirus, an adeno-associated virus (AAV), and a lentivirus.

In another embodiment of any of the above methods, the composition comprises a mesenchymal stem cell (MSC), expressing exogenous osteoprotegerin (OPG) and the MSC is a bone marrow derived MSC. In some embodiments, the composition comprises a mesenchymal stem cell (MSC), expressing exogenous osteoprotegerin (OPG) wherein the MSC is a bovine MSC, a feline MSC, a canine MSC, a murine MSC, an equine MSC, or a human MSC. In some embodiments, the MSC is a human MSC. In some embodiments, the human MSC has a cell marker profile comprising CD105⁺, CD166⁺, CD29⁺, CD44⁺, CD14⁻, CD34⁻, and CD45⁻. In some embodiments the composition comprises a mesenchymal stem cell (MSC), expressing exogenous osteoprotegerin (OPG) wherein the MSC is osteogenically differentiated.

Administration of Additional Therapeutic Agents

The methods disclosed herein can further comprise, or alternatively consist essentially of, or yet further consist of administration of an effective amount of additional therapeutic agents to augment or enhance the therapeutic efficacy of the disclosed methods. Non-limiting examples of additional therapeutic agents to augment or enhance the therapeutic efficacy of the disclosed methods include bone morphogenic protein (BMP), growth factors, IGF-I, IGF-II, platelet-derived growth factor, basic and acidic fibroblast growth factor (FGF), BMP2, BMP4, OP-1, FGF1, FGF2, TGF-γ1, TGF-β2, TGF-β3, Collagen 1, laminin 1-6, fibronectin, parathyroid hormone related peptide (PTHrP), vitronectin, etidronate, clodronate, alendronate, pamidronate, risedronate, zoledronate, hydroxyapatite, hyaluronic acid, prednisone, budesonide, prednisolone, cyclosporine, tacrolimus, sirolimus, everolimus, azathioprine, leflunomide, mycophenolate, abatacept, adalimumab, anakinra, certolizumab, etanercept, golimumab, infliximab, ixekizumab, natalizumab, rituximab, secukinumab, secukinumab, tocilizumab, ustekinumab, vedolizumab, basiliximab, daclizumab, muromonab, teriparatide and chitosan. The compositions may be supplemented with exogenous testosterone, dihydrotestosterone, estrogens, estradiol, GH/IGF-1, thyroid hormone, parathyroid hormone, calcitonin, glucocorticoids, cortisol and vitamin D.

The following examples are provided to illustrate but not limit the invention.

### EXAMPLES

### Example 1: Osteoprotegerin-Mediated Osteoclast Inhibition Is Augmented On Nanoparticulate Mineralized Collagen Glycosaminoglycan Materials

Characterization of the instructive capabilities of extracellular matrix (ECM)-inspired materials for osteoprogenitor differentiation has sparked questions on the interactions between such materials and the host microenvironment. In one aspect of this disclosure, adenoviral mediated expression of OPG (AdOPG), and an endogenous osteoclast inhibitor against RANKL in primary human mesenchymal stem cells (hMSCs) with a highly osteogenic MC-GAG scaffold for osteoclast inactivation in augmentation of bone regeneration, is provided. AdOPG demonstrated no effects on the viability, proliferation, osteogenic gene expression, activation of intracellular signaling molecules, or mineralization of hMSCs. hMSCs differentiated on MC-GAG expressed a lower ratio of endogenous RANKL/OPG protein on MC-GAG compared to a non-mineralized collagen glycosaminoglycan (Col-GAG) scaffold. While AdOPG demonstrated no effects on hMSC viability or osteogenic differentiation, AdOPG-transduction significantly reduced the RANKL/OPG ratio for both mineralized and non-mineralized scaffolds. A co-culture system was used to understand the interplay between simultaneously differentiating hMSCs and primary human pre-osteoclasts (hOCs).

hMSCs augmented hOC-mediated resorption and hOCs augmented hMSC-mediated mineralization suggesting that stimulatory effects exist between the cell types when both are in the process of undergoing differentiation. While AdOPG-transduction diminished hOC-mediated resorption, the stimulatory effects of hOCs on hMSC-mediated mineralization were unaffected. Notably, AdOPG-transduced hMSCs reduced the resorptive activity of osteoclasts with a greater effect on MC-GAG compared to Col-GAG. AdOPG-transduced hMSCs co-cultured with hOCs also expressed higher levels of phosphorylated Smad1/5, phosphorylated ERK1/2, and Runx2 on MC-GAG compared to Col-GAG. Taken together, the addition of osteoprotegerin to MC-GAG-mediated hMSC osteogenic differentiation simultaneously diminishes osteoclast resorptive capacity without affecting the positive regulatory effects on osteogenic differentiation.

Materials and Methods

Pit Assay/Quantification, ELISA Fabrication and chemical crosslinking of non-mineralized and mineralized collagen scaffolds: Col-GAG and MC-GAG scaffolds were prepared using lyophilization [31-33]. Briefly, microfibrillar, type I collagen (Collagen Matrix, Oakland, N.J.) and chondroitin-6-sulfate (Sigma-Aldrich, St. Louis, Mo.) were combined in suspension in the absence and presence of calcium salts (calcium nitrate hydrate: Ca(NO₃)₂.4H2O; calcium hydroxide: Ca(OH)₂, Sigma-Aldrich, St. Louis, Mo.) in an acetic acid (Col-GAG) or phosphoric acid (MC-GAG) solution. Using a constant cooling rate technique at a rate of 1° C./min, the solution was frozen from room temperature to −10° C. using a freeze dryer (Genesis, VirTis). Following sublimation of the ice phase, scaffolds were sterilized via ethylene oxide and cut into 8 mm disks for culture.

Crosslinking of scaffolds was performed after rehydration in phosphate buffered saline (PBS) overnight or at least 4 hr using 1-ethyl-3-(3-dimethylaminopropyl) carbodiimide (EDAC, Sigma-Aldrich) and N-hydroxysuccinimide (NHS, Sigma Aldrich) at a molar ratio of 5:2:1 EDC:NHS:COOH where COOH represents the amount of collagen in the scaffold [34]. Scaffolds were washed with PBS to remove any of the residual chemical.

Cell culture: Primary human mesenchymal stem cells (hMSCs, Lonza, Inc., Allendale, N.J.) were expanded in proliferation media composed of Dulbecco's Modified Eagle Medium DMEM (Corning Cellgro, Manassas, Vt.) supplemented with 10% fetal bovine serum (FBS) (Atlanta Biologicals, Atlanta, Ga.), 2 mM L-glutamine (Life Technologies, Carlsbad, Calif.), 100 IU/mL penicillin/100 μg/mL streptomycin (Life Technologies). 2D culture: hMSCs of passage 3-5 were plated at 5000 cells per well in 12 well plates, grown until 80-90% confluent, and then transduced with and without an adenovirus expressing OPG and RFP (AdOPG) in DMEM at a multiplicity of infection (MOI) of 200 and 4 μg/mL of polybrene (Sigma-Aldrich, St. Louis, Mo.). 24 h after transduction, hMSCs were subjected to differentiation medium consisting of proliferation media plus 10 mM β-glycerophosphate, 50 μg/mL ascorbic acid and 0.1 μM dexamethasone. Cell cultures were evaluated on day 7 post-transduction for morphological changes, transduction efficiency, and western blot.

Osterogenic differentiation of hMSCs on Col-GAG and MC-GAG: 3×10⁵ hMSCs were seeded onto 8 mm discs of CG-GAG and MC-GAG scaffolds in proliferation media. 24 h after seeding, media was switched to osteogenic differentiation media consisting of 10 mM (3-glycerophosphate, 50 μg/mL ascorbic acid and 0.1 μM dexamethasone.

Indirect hMSC and hOC co-cultures: 2×10⁵ hMSCs were seeded to 6 mm Col-GAG and MC-GAG scaffolds in proliferation media. 24 h after seeding hMSCs, 6×10⁴ primary human osteoclast precursors (hOCs; Lonza, Inc., Allendale, N.J.) were cultured in Osteoclast Precursor Basal Medium (Lonza, Allendale N.J.) supplemented with 33 ng/mL macrophage-colony stimulating factor (M-CSF), 66 ng/mL of RANKL, 10 mM (3-glycerophosphate, 50 μg/mL ascorbic acid, 0.1 μM dexamethasone for concurrent hMSC and hOC differentiation on 24 well Corning Osteo Assay Surface Microplates (Corning, N.Y.). After 2 h, Col-GAG and MC-GAG scaffolds were transferred to 8 μm Transwell inserts (Corning, N.Y.) and co-cultured with hOCs. Media were changed every 3 days for 3 weeks.

Direct hMSC and hOC co-cultures: 3.5×10⁵ hMSCs were seeded to 8 mm Col-GAG and MC-GAG scaffolds in proliferation media. 24 h after seeding hMSCs, 6×10⁴ hOCs were cultured in Osteoclast Precursor Basal Medium (Lonza, Allendale N.J.) supplemented with 33 ng/mL M-CSF, 66 ng/mL RANKL, 10 mM β-glycerophosphate, 50 μg/mL ascorbic acid, 0.1 μM dexamethasone on 24 well Osteo Assay Microplates. After 2 h, Col-GAG and MC-GAG scaffolds were transferred to the Osteo Assay Plates and directly co-cultured with hOCs. Media were changed every 3 days for 2 weeks.

Quantitative real-time reverse-transcriptase polymerase chain reaction: Total RNeasy kit (Qiagen, Valencia, Calif.) was used to extract RNA from scaffolds at 0, 3, and 14 days of culture. Gene sequences for 18S, runt-related transcription factor 2 (Runx2), osteopontin (OPN), osteoprotegerin (OPG) and Receptor activator of nuclear factor kappa-B ligand (RANKL) were obtained from the National Center for Biotechnology Information gene database and primers were designed (Table 1). Quantitative real-time reverse-transcriptase polymerase chain reactions (RT-PCR) were performed on the Opticon Continuous Fluorescence System (Bio-Rad Laboratories, Inc., Hercules, Calif.) using the QuantiTect SYBR Green QPCR kit (Qiagen). Cycle conditions were as follows: reverse transcription at 50° C. (30 min); activation of HotStarTaq DNA polymerase/inactivation of reverse transcriptase at 95° C. (15 min); and 45 cycles of 94° C. for 15 s, 58° C. for 30 s, and 72° C. for 45 s. Results were analyzed and presented as representative graphs of triplicate experiments.

TABLE 1
Primer Sequences

| Genes | Oligonucleotide sequence |
|---|---|
| 18S sense | 5′-TAGAGTGTTCAAAGCAGGCCCG-3′ |
| 18S antisense | 5′-TCCCTCTTAATCATGGCCTCAG-3′ |
| ALP sense | 5′-AAGCCGGTGCCTGGGTGGCCAT-3′ |
| ALP antisense | 5′-ACAGGAGAGTCGCTTCAGAG-3′ |
| Runx2 sense | 5′-TCGGAGAGGTACCAGATGGG-3′ |
| Runx2 antisense | 5′-AACTCTTGCCTCGTCCACTC-3′ |
| BSP II sense | 5′-GGACTGCCAGAGGAAGCAAT-3′ |
| BSP II antisense | 5′-GGCCTGTACTTAAAGACCCCA-3′ |

ALP, alkaline phosphatase; Runx2, runt-related transcription factor 2; BSP II, bone sialoprotein II

ELISA: Supernatants were collected from 9hMSC only, osteoclast only, or hMSC and hOC co-cultures. OPG protein concentrations were determined using the human OPG DuoSet ELISA kit (R&D Systems, Minneapolis, Minn.) according to manufacturer's instructions. Briefly, a 96-well microplate was coated with the capture antibody and incubated overnight at room temperature. After blocking, samples were incubated for 2 hours at room temperature with the detection antibody, followed by incubation with streptavidin-horseradish peroxidase (HRP) for 20 min. The reaction was stopped by adding 100 μL of 2N H₂SO₄. Plates were read at 450 nm and 540 nm wavelengths on the Epoch microplate reader (BioTex, Winooski, Vt.).

Microcomputed tomographic (microCT) imaging: Scaffolds were fixed using 10% formalin and mineralization was quantified by micro-computed tomographic imaging (microCT) using the Scanco 35 (Scanco Medical AG, Bruttisellen, Switzerland) in triplicate for each time point. Scans were performed at medium resolution with a source voltage of 70 E (kVp) and I (pA) of 114. The images had a final element size of 12.5 μm. Images were analyzed using software supplied from Scanco (Image Processing Language version 5.6) and reconstructed into three-dimensional (3D) volumes of interest. Optimum arbitrary threshold values of 20 (containing scaffold and mineralization) and 80 (containing mineralization alone) were used uniformly for all specimens to quantify mineralized areas from surrounding unmineralized scaffold.

Analysis of 3D reconstructions was performed using Scanco Evaluation script #2 (3D segmentation of two volumes of interest: solid dense in transparent low-density object) and script #6 (bone volume/density only bone evaluation) for volume determinations.

Western blot: Lysates were prepared from scaffolds at 0, 3, 14, 28, 42 and 56 days of culture using SDS sample buffer and equal amounts were subjected to 4-20% SDS-PAGE (Bio-Rad, Hercules, Calif.). Western blot analysis was carried out with antibodies against phosphorylated small mothers against decapentapalegicl/5 (p-Smad1/5), total Smad5, phosphorylated extracellular regulated kinasel/2 (p-ERK1/2), total ERK1/2, and β-actin followed by 1:4000 dilutions of horseradish peroxidase-conjugated IgG antibodies (Bio-Rad, Hercules, Calif.) and an enhanced chemiluminescent substrate (Thermo Scientific, Rockford, Ill.). For detection of p-Smad1/5 and total Smad5, 10 μg of lysate was loaded per lane. For detection of p-ERK1/2 and total ERK1/2, 20 μg of lysate was loaded per lane. All primary phospho-antibodies were obtained from Cell Signaling Technologies (Beverly, Mass.). β-actin antibody was obtained from Santa Cruz Biotechnology (Santa Cruz, Calif.). Imaging analysis was carried out using ImageJ (NIH, Bethesda, Md.). The RANKL/OPG relative protein ratios were calculated by quantifying the densitometry of all RANKL and OPG normalized to actin using Image J (NIH, Bethesda, Mass.).

Water Soluble Tetrazolium-1 (WST-1) Assay: Culture media was supplemented with cell proliferation reagent WST-1 (Roche, Basel, Switzerland) at a 1:10 concentration. Scaffolds were incubated for 3-4 h at 37° C. in a humidified atmosphere with 5% CO₂. Absorbance of the incubation medium was measured at 450 and 690 nm (Epoch spectrophotometer, BioTek, Winooski, Vt.).

Tartrate-Resistant Acid Phosphatase (TRAP) Staining: hOCs were detected using Leukocyte TRAP Kit 387-A (Sigma-Aldrich) according to the manufacturer's instructions. Briefly, cultured cells were fixed with formaldehyde for 5 min at room temperature, washed, and air-dried. After staining, TRAP-positive multinucleated cells were observed under a phase-contrast microscope at 20× magnification and digitally photographed.

Resorption Pit Assay: Activity of hOCs in single culture or co-cultured with scaffolds with and without hMSCs were evaluated for resorption pit formation on Osteo Assay microplates. At the completion of the culture period, culture media was aspirated and 5004 of 10% bleach solution was added for 5 minutes at room temperature. The wells were washed with distilled water and allowed to dry at room temperature for 3-5 h. Pits were observed using a standard microscope digitally photographed. Percentage of resorption for the whole well of the culture at magnification 2× was calculated by ImageJ.

Statistical analysis: All statistical analyses were performed using SPSS Version 24 (Chicago, Ill.). Data points were composed of duplicates of at least three independent experiments, unless otherwise indicated. Mean measurements of mRNA expression were analyzed for statistical significance by analyses of variance (ANOVA) followed by post hoc tests using the Tukey criterion. A value of p<0.05 was considered significant.

Results

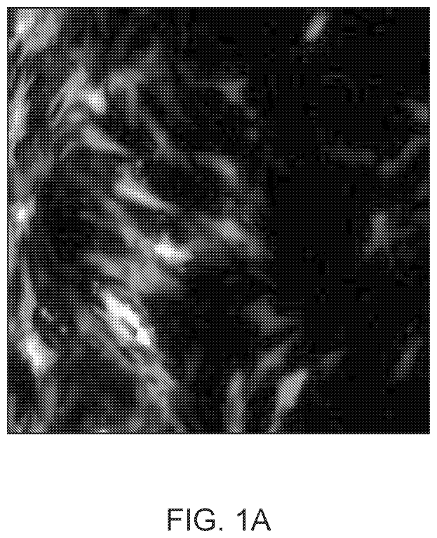

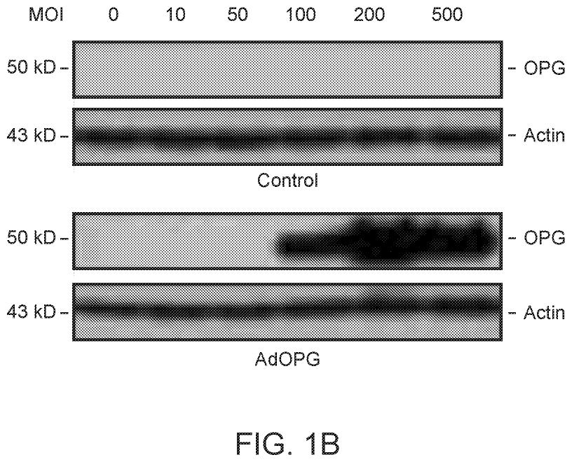

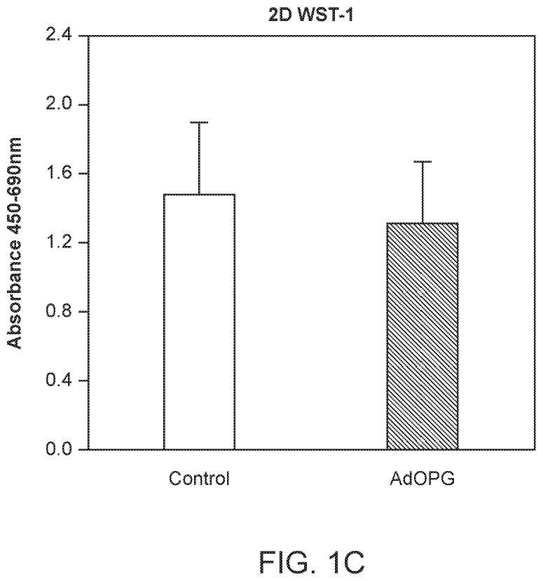

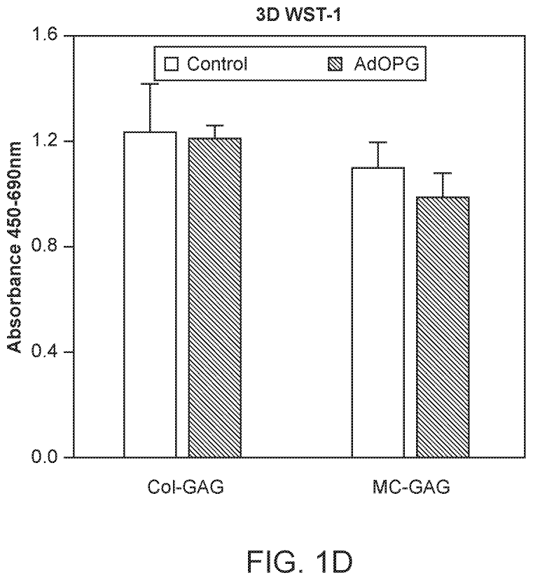

AdOPG transduction of primary hMSCs differentiated on Col-GAG and MC-GAG does not affect cell viability or proliferation: Applicant hypothesized that a prolongation of OPG expression may augment the anti-resorptive capabilities of MC-GAG. To augment anti-osteoclastogenic activities induced by MC-GAG, primary bone marrow-derived hMSCs (CD105+CD166+CD29+CD44+CD14-CD34-CD45-) were transduced with adenoviruses expressing OPG (AdOPG). Control and AdOPG transduced hMSCs were cultured in osteogenic differentiation medium for 7 and 14 days and evaluated for infection efficiency, OPG expression, and effects on cell viability and proliferation (FIG. 1A). Using the co-expressed red fluorescent protein (RFP) as an indicator, AdOPG transduction resulted in a 35% infection efficiency based on cell counting with a maximal amount of protein expression on western blot analysis at a multiplicity of infection (MOI) of 200 (FIG. 1B).

Prior to analysis of long term scaffold mineralization, the viability and proliferation of hMSCs were confirmed by measuring the mitochondrial dehydrogenase activity using the WST-1 tetrazolium salt colorimetric assay. hMSC viability and proliferation were not found to be significantly different in control versus AdOPG-infected cells in two dimensional cultures after three weeks of transduction (FIG. 1C). To confirm that cells were viable in long term three-dimensional cultures, control and AdOPG were seeded on Col-GAG and MC-GAG and differentiated for 8 weeks in osteogenic differentiation medium (FIG. 1D). Again, hMSC cell viability and proliferation were found to be equivalent on control and AdOPG hMSCs on Col-GAG or MC-GAG with no statistically significant differences between the materials.

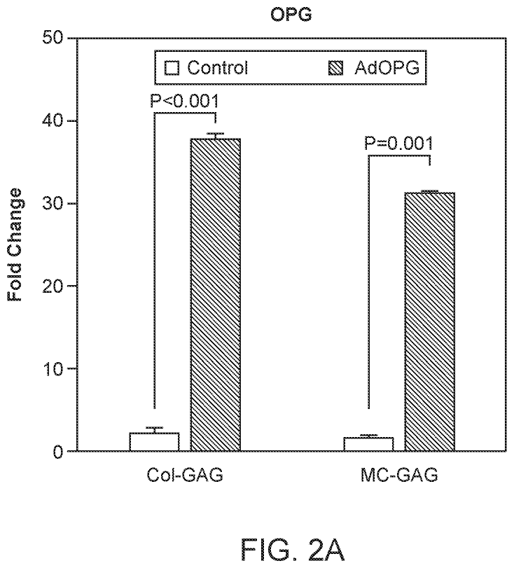

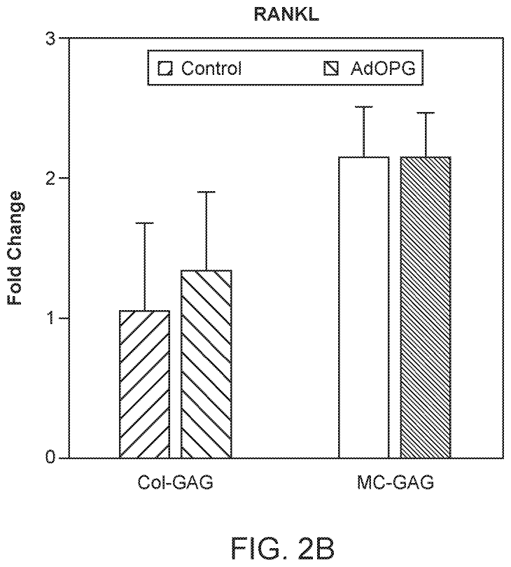

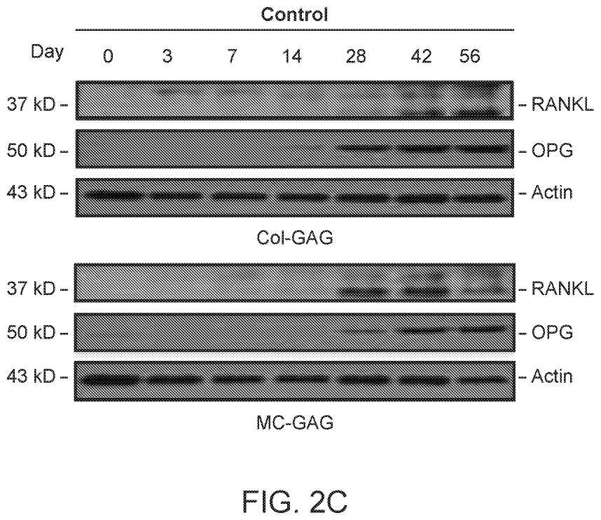

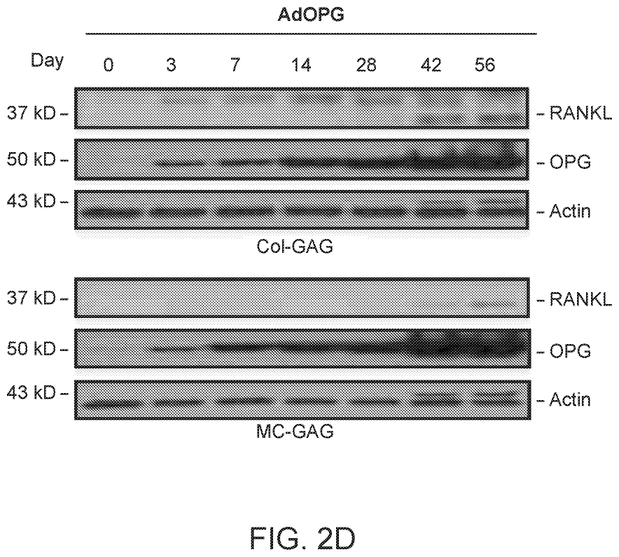

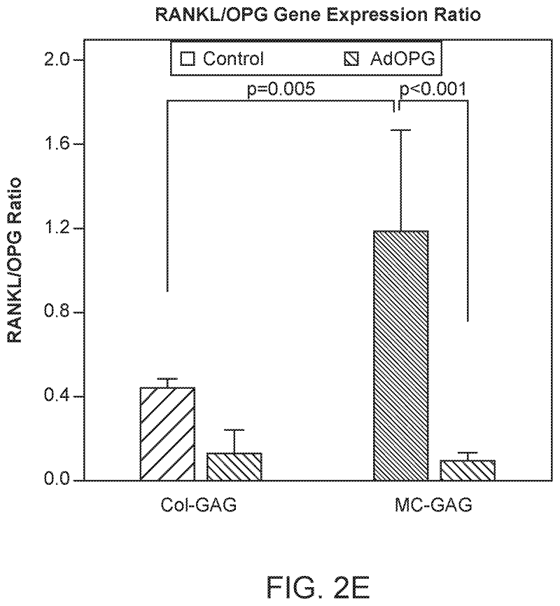

AdOPG transduction changes RANKL/OPG relative protein ratios in primary hMSCs differentiated on Col-GAG and MC-GAG: The relative expression of OPG to RANKL in control and AdOPG-infected cells was evaluated (FIG. 2). Control and AdOPG-infected cells were induced to undergo osteogenic differentiation on Col-GAG and MC-GAG for 14 days and QPCR was performed to assess OPG and RANKL gene expression (FIG. 2A and FIG. 2B). No statistically significant differences were found in OPG or RANKL expression between control cells on either material. In the presence of AdOPG, OPG gene expression increased over 30-fold in cells cultured on either scaffold while no differences in RANKL expression was noted.

Protein expression was next evaluated using Western blot analysis of OPG and RANKL expression over 56 days (FIG. 2C and FIG. 2D). Two dominant isoforms of RANKL were detected including a band at 35 kDa as well as a higher molecular weight band near 45 kDa which may reflect expression of different splice variants. Densitometry was utilized to quantify the relative expression of OPG and RANKL and the RANKL/OPG protein expression ratio over 56 days was evaluated (FIG. 2F). A statistically significant difference was found in the RANKL/OPG protein expression ratios between the groups [F(3,24)=19.35, p<0.001]. In both Col-GAG and MC-GAG, AdOPG expression lowered the RANKL/OPG protein expression ratio as expected (p<0.001). Col-GAG displayed a higher RANKL/OPG ratio compared to MC-GAG in control cells (p<0.001). Taken together, MC-GAG induces diminished RANKL/OPG gene expression ratio compared to Col-GAG. In both materials, the addition of AdOPG changes the RANKL/OPG ratio to favor OPG significantly compared to RANKL.

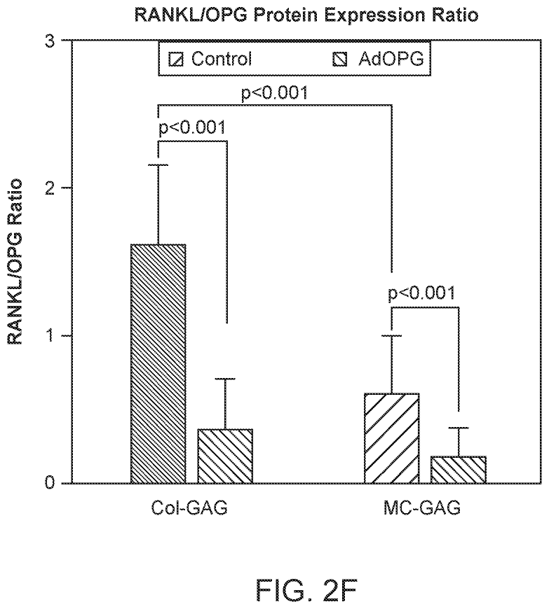

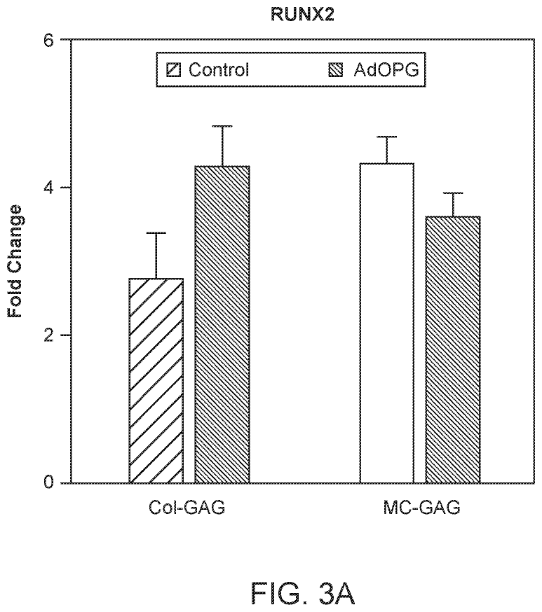

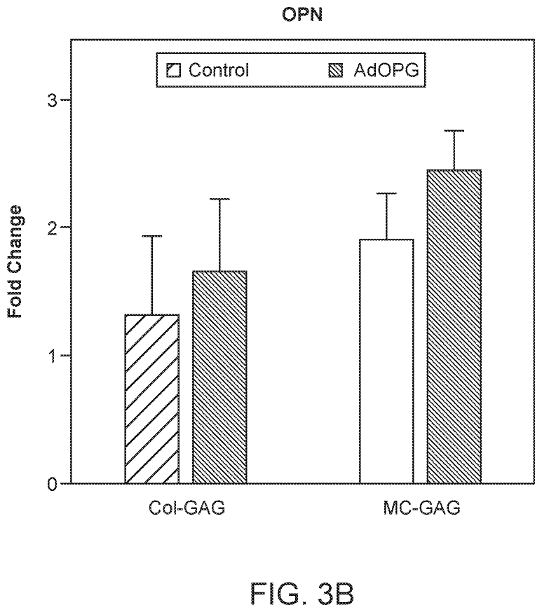

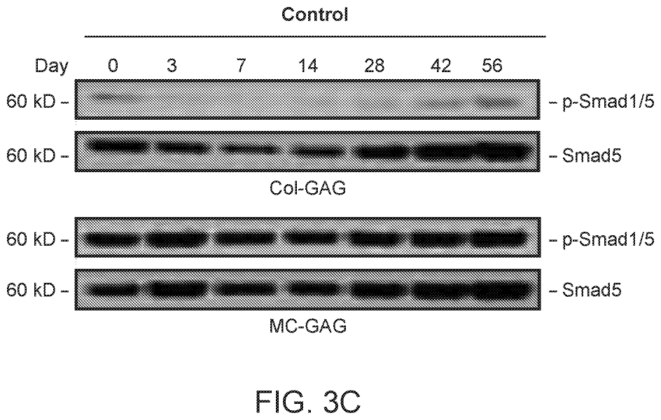

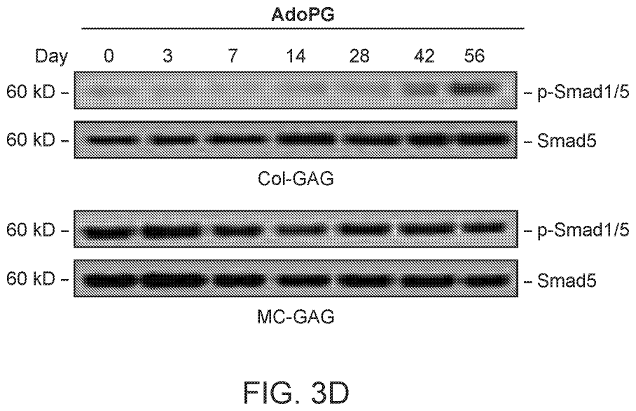

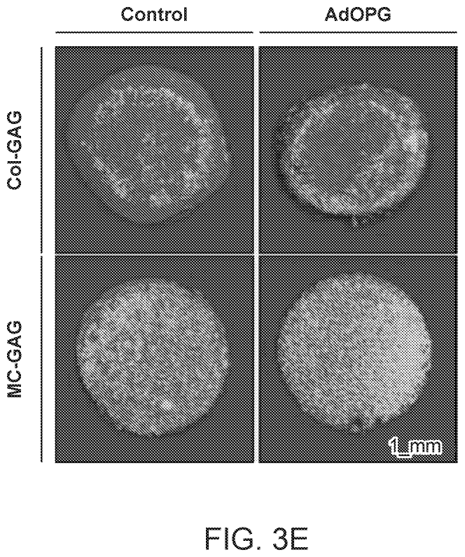

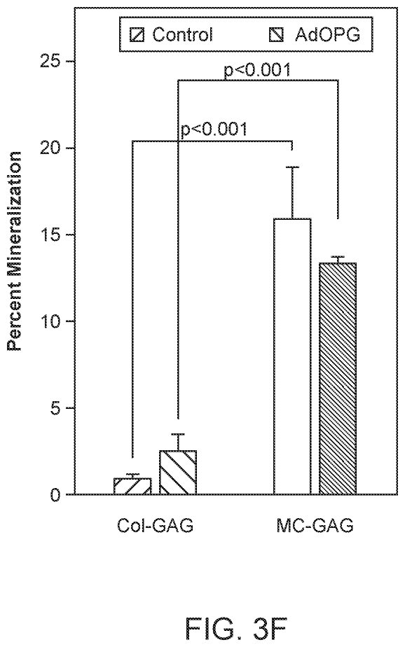

AdOPG does not affect hMSC mineralization on Col-GAG or MC-GAG: To evaluate whether AdOPG directly affects mineralization in the system of this Example, control and AdOPG-infected hMSCs undergoing osteogenic differentiation on Col-GAG and MC-GAG were evaluated for expression of osteogenic markers, activation of osteogenic signaling pathways, and matrix mineralization (FIGS. 3A-3F). At 14 days of culture, no significant differences in runt-related transcription factor 2 (RUNX2) or osteopontin (OPN) gene expression were found between control and AdOPG cells on Col-GAG or MC-GAG (FIGS. 3A and B).

To evaluate the contribution of AdOPG to the activation of intracellular signaling pathways that contribute to mineralization on Col-GAG and MC-GAG, Western blots analyses of control and AdOPG-infected hMSCs undergoing osteogenic differentiation on Col-GAG or MC-GAG were performed on total protein lysates over 8 weeks (FIG. 3C and FIG. 3D). No significant differences in p-Smad1/5 activations were detected in the absence or presence of AdOPG. MC-GAG, induces significantly more p-Smad1/5 compared to Col-GAG.

Matrix mineralization was also quantified using micro-CT analysis (FIG. 3E and FIG. 3F). Again, no significant differences between control and AdOPG hMSCs were detected on either Col-GAG and MC-GAG. MC-GAG demonstrated more mineralization than Col-GAG with or without AdOPG.

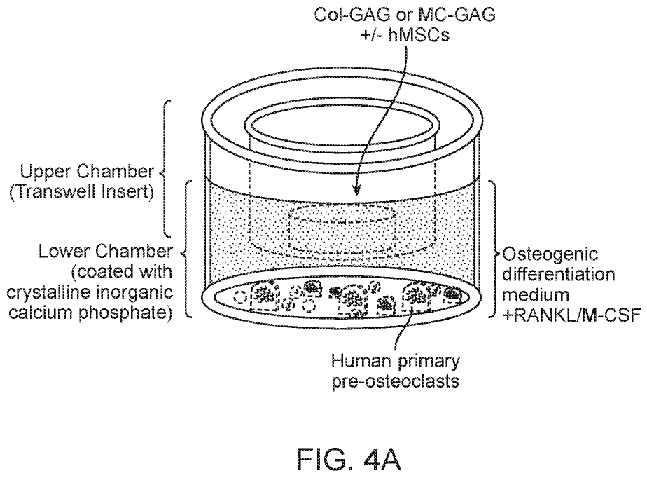

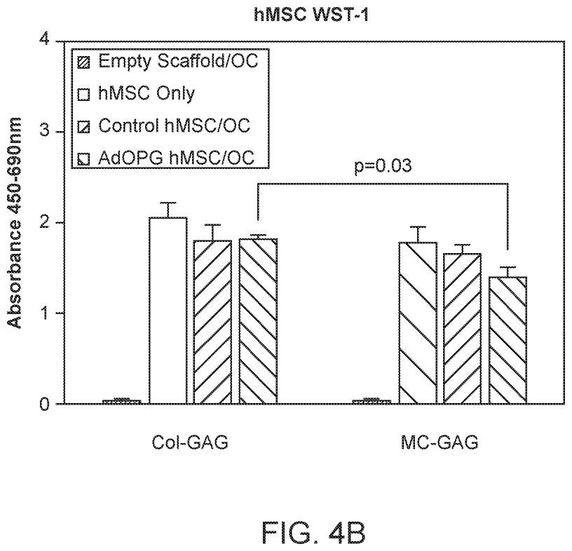

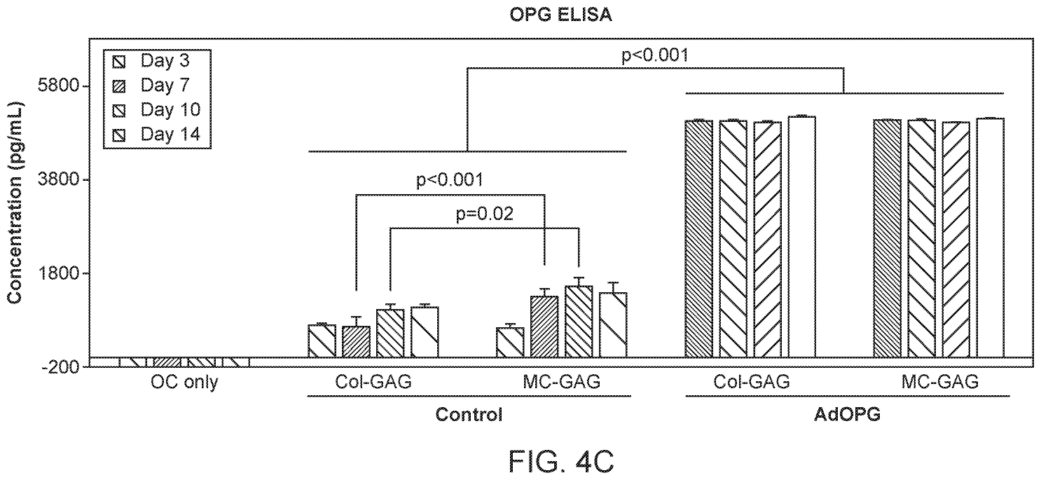

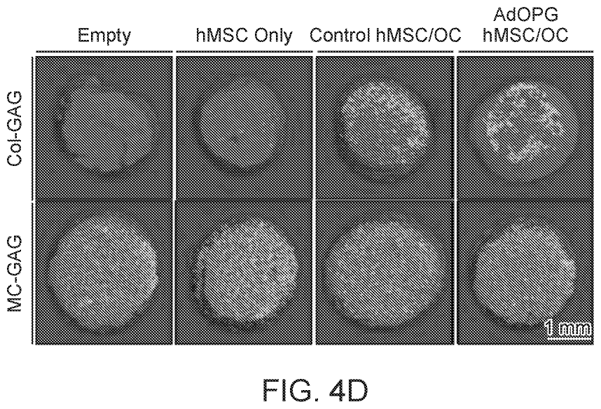

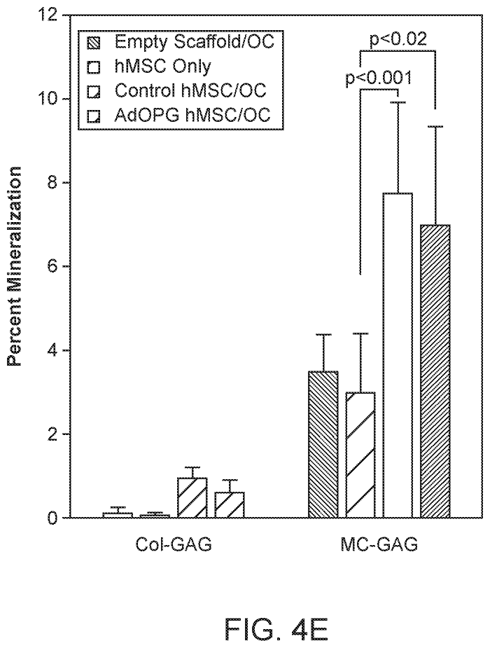

Indirect Osteoclast co-cultures augment mineralization in hMSCs undergoing mineralization on MC-GAG in the absence or presence of AdOPG: To understand the effects of MC-GAG on human osteoclasts, two co-culture techniques were employed: indirect and direct. Indirect co-cultures were performed to isolate the effects of hOCs on hMSCs and vice versa without the confounding effects of scaffold resorption from direct contact and to understand the paracrine effects between the two cell types (FIG. 4A). Direct co-cultures were devised for the purposes of understanding the net effects of the system with cells and materials in direct contact with each other. For indirect co-cultures, Col-GAG or MC-GAG scaffolds were cultured in an 8 μm Transwell insert with and without hMSCs seeded on the materials (upper chamber). In the lower chamber, human primary pre-osteoclasts were seeded on a calcium phosphate coated plate where resorptive activity may be evaluated. Co-cultures were concurrently differentiated with medium supplemented with RANKL, M-CSF, β-glycerophosphate, ascorbic acid, and dexamethasone.

In the indirect co-culture system, the effects on hMSCs were first evaluated. After 21 days of culture, the Transwell inserts (upper chambers) were subjected to WST-1 assay (FIG. 4B). Statistically significant differences in viability and proliferation were found between hMSCs of the different groups [F (7,18)=81.36, p<0.001]. Empty Col-GAG or MC-GAG scaffolds without hMSCs co-cultured with osteoclasts displayed no evidence of cell viability or proliferation as expected (p<0.001 compared to any other condition). No differences were found between hMSCs cultured without osteoclasts (hMSCs Only) or control hMSCs co-cultured with osteoclasts (Control hMSC/OC) on either material. With AdOPG, hMSCs cultured on MC-GAG demonstrated a decrease in viability and proliferation compared to Col-GAG in a statistically significant fashion (p=0.03).

To determine the amount of soluble OPG in the co-culture system, ELISAs were performed over the entirety of the co-culture period and compared to an osteoclast only negative control (FIG. 4C). Differences between the cultures were found to be statistically significant [F (4,15)=552.37, p<0.001]. Osteoclasts did not display any significant amount of OPG secretion as expected. In control cells, hMSCs on MC-GAG produced significantly more endogenous OPG compared to Col-GAG at days 7 (p<0.001) and 10 (p=0.02). Multiple comparisons of any control timepoint versus any AdOPG-infected timepoint for either material displayed significantly higher amounts of OPG in AdOPG-infected cells (p<0.001 for all conditions).

Mineralization was evaluated after 3 weeks of co-culture using micro-CT scanning (FIG. 4D and FIG. 4E). Overall, differences in mineralization were found to be present [F(7,26)=26.48, p<0.001]. An increase in osteogenic differentiation occurred in control hMSC co-cultures hOCs (Control hMSC/OC) compared to hMSCs single cultures (hMSC only) on MC-GAG materials (p<0.001). Although a mild increase in mineralization was evident qualitatively and quantitatively on Col-GAG in co-cultures versus single cultures, this difference did not reach statistical significance. In MC-GAG, the increase in mineralization for co-cultures with AdOPG compared to hMSC only single cultures remained significant (p=0.02). In combination, indirect co-cultures of differentiating hMSCs with hOCs resulted in positive regulation of osteogenic differentiation manifested by mineralization, particularly on MC-GAG. This increase in mineralization is largely unaffected by AdOPG transduction.

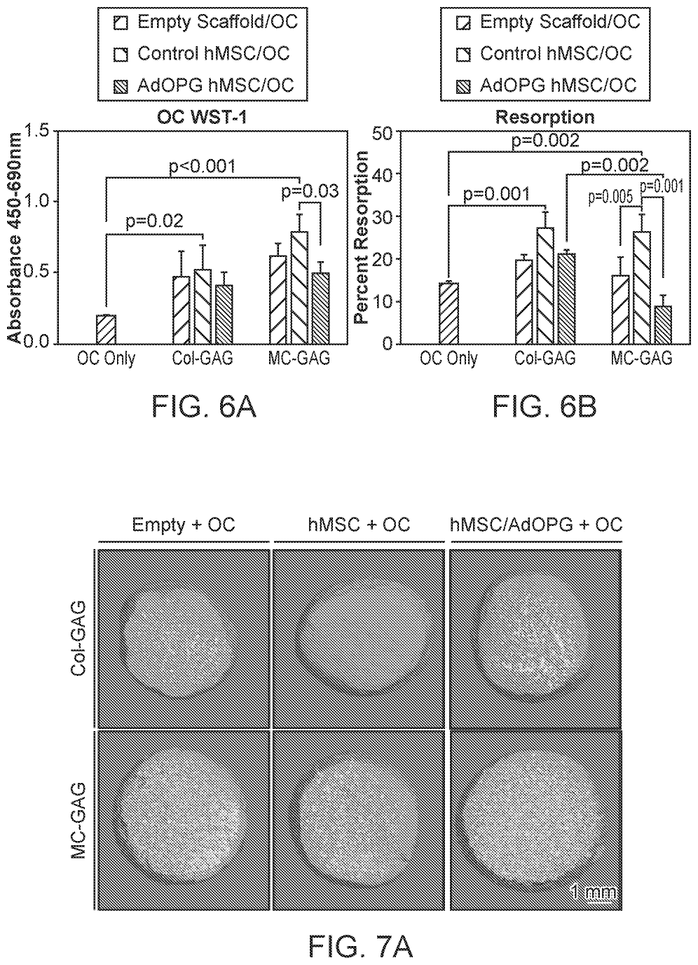

Indirect co-cultures with AdOPG-transduced hMSCs on MC-GAG diminish hOC resorptive activity: In the same indirect co-cultures, the effects on osteoclasts were also evaluated (FIGS. 6A and 6B). Following removal of the Transwell inserts, the lower chamber consisting of hOCs were subjected to WST-1 assay and found to have statistically significant differences on ANOVA [F (6,21)=9.23, p<0.001]. An increase in viability and proliferation or hOCs occurred in co-cultures with control hMSCs on Col-GAG or MC-GAG (p=0.02 and p<0.001, respectively). AdOPG-transduced hMSCs diminished viability and proliferation of hOCs compared to control hMSCs on MC-GAG (p=0.03). Interestingly, minimal differences in hOC viability were detected on Col-GAG with hMSCs transduced with AdOPG.

Resorptive activity of the hOCs was also characterized and found to have significant differences via ANOVA [F(6,17)=15.34, p<0.001] (FIG. 6B). hOC-mediated resorption increased significantly in co-cultures of hMSCs on Col-GAG (p=0.001) or MC-GAG (p=0.002) compared to hOC single cultures. In AdOPG transduced hMSCs, hOC-mediated resorption diminished on both materials compared to control hMSCs, however only the decrease in MC-GAG reached statistical significance (p<0.001). In combination, these data suggest that differentiating hMSCs increase the viability, proliferation, and resorptive capabilities of hOCs on either non-mineralized or mineralized collagen glycosaminoglycan materials. While co-cultures with AdOPG-transduced hMSCs mildly reduced hOC viability, proliferation, and resorption on Col-GAG, MC-GAG demonstrated a significantly greater effect.

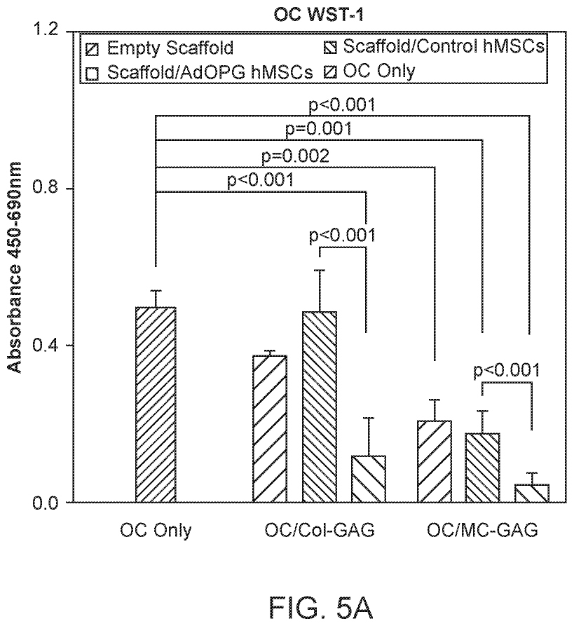

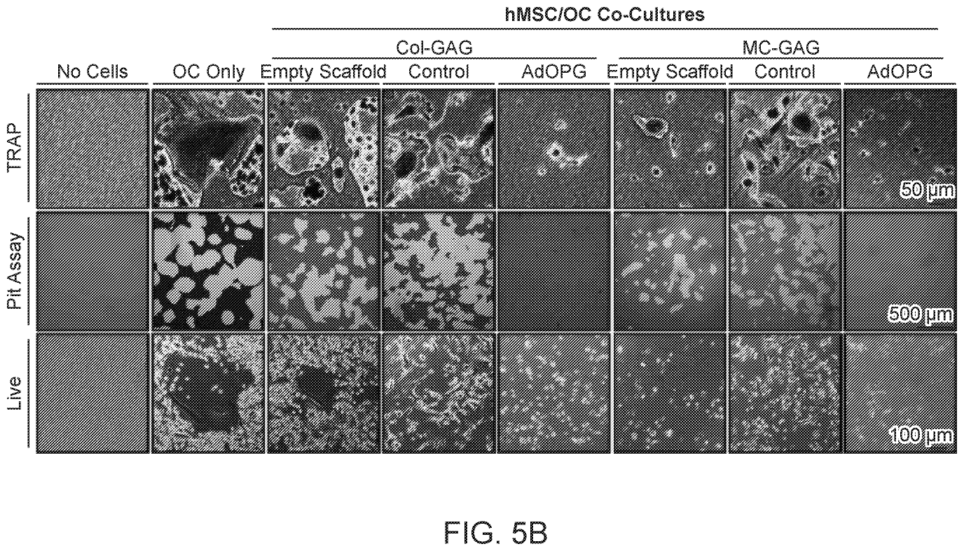

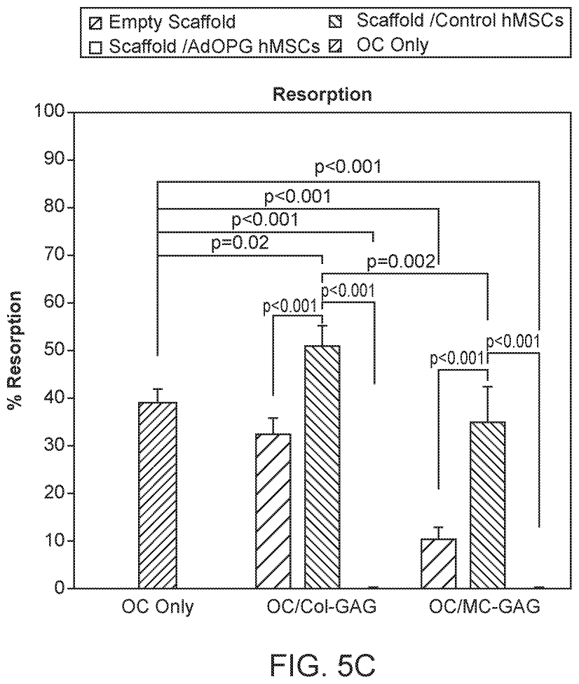

Direct contact of transduced hMSCs on Col-GAG and MC-GAG diminishes the proliferation and resorption activity of osteoclasts: Next, the effects on osteoclasts were evaluated in the direct co-culture system described above. hOCs were first plated on a calcium phosphate coated plate. Two hours after seeding, hMSCs seeded on Col-GAG or MC-GAG was transferred to each well in direct contact with hOCs. Co-cultures were simultaneously differentiated with media supplemented with RANKL, M-CSF, β-glycerophosphate, ascorbic acid, and dexamethasone. After 14 days of culture, the respective scaffolds were removed and osteoclasts were subjected to WST-1 assay (FIG. 5A). Statistically significant differences between the cultures were noted [F (6,16)=23.82, p<0.001]. In the presence of MD Col-GAG, the viability and proliferation were both diminished in the presence of AdOPG.

Osteoclast differentiation and activity were assessed using both TRAP staining and resorption pit assays (FIG. 5B and FIG. 5C). In all conditions with the exception of the negative control, TRAP activity was detected indicating that active osteoclasts were present. Osteoclast activity, detected by resorption pits of the inorganic crystalline calcium phosphate coating of the plate, demonstrated significant differences between the groups. In the presence of empty Col-GAG, a mild decrease in resorption was elicited which was rescued with the addition of control hMSCs. In MC-GAG, a significant decrease in resorptive abilities was seen, which was also improved with the addition of control hMSCs. In both cases, osteoclast resorption was completely inhibited when transduced with AdOPG.

Quantification of the total resorption pit areas demonstrated significant differences in resorption activity between the conditions [F(6,17)=15.34, p<0.001] (FIG. 5C). In posthoc comparisons, no statistically significant differences were seen between osteoclasts cultured alone (OC Only) and osteoclasts co-cultured with empty Col-GAG whereas osteoclasts cultured with empty MC-GAG were significantly less active (p<0.001). In co-cultures with control hMSCs differentiated on Col-GAG or MC-GAG, resorption increased compared to empty saffolds (p<0.002), although the quantity of resorption continued to be lower in MC-GAG compared to Col-GAG. In the presence of AdOPG, resorption was completely eliminated for either material compared to scaffolds with control hMSCs. (p<0.002) for both.

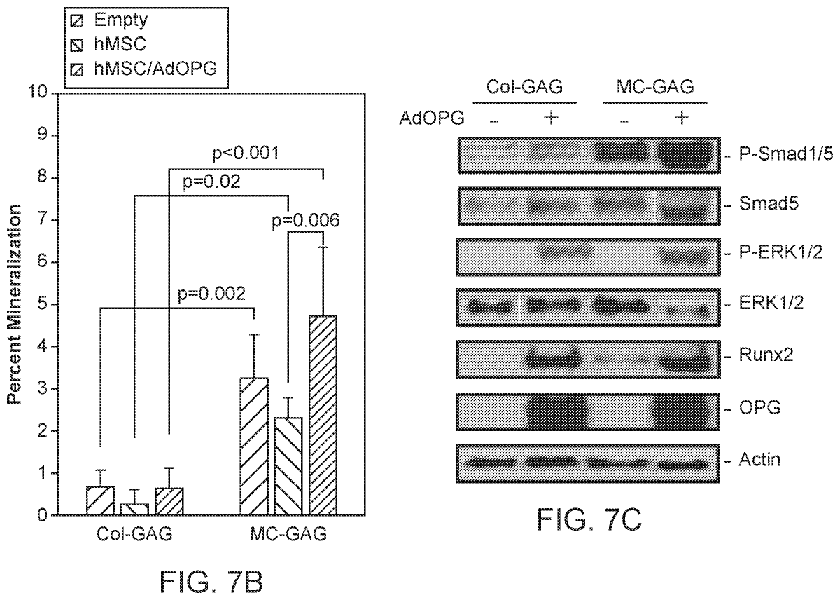

AdOPG transduction augments mineralization and hMSC expression of phosphorylated Smad1/5, Runx2, and phosphorylated ERK1/2 when directly contacting osteoclasts: Direct contact of hMSCs differentiated on Col-GAG and MC-GAG with hOCs allows for investigation of the net effects of positive and negative regulation including resorption on mineralization. Empty Col-GAG and MC-GAG scaffolds, scaffolds seeded with control hMSCs or AdOPG-transduced hMSCs were directly co-cultured with hOCs and concurrently differentiated for 14 days. Scaffolds were assessed for mineralization and activation of intracellular mediators known to be involved in osteogenic differentiation (FIGS. 7A-7C).

Unlike indirect co-cultures, direct co-cultures with hOCs resulted in a decrease in mineral content on both Col-GAG and MC-GAG with control hMSCs when compared to empty scaffolds (FIGS. 7A and 7B). The decrease in mineralization is in concordance with the increase in hOC activity (FIGS. 5B and 5C) seen in the presence of hMSCs. When hMSCs transduced with AdOPG on Col-GAG and MC-GAG were directly co-cultured with hOCs, mineralization was significantly improved resulting in a net osteogenic state.

To compare the osteogenic mechanisms activated in hMSCs in the direct co-culture system, intracellular mediators known to be upregulated in osteogenic differentiation were evaluated on western blot analysis (FIG. 7C). Interestingly, unlike single cultures with hMSCs, direct co-cultures resulted in an upregulation of p-Smad1/5, p-ERK1/2, and Runx2. These data suggest that hOCs simultaneously positively regulate hMSC osteogenesis while actively resorbing mineralized volumes. More importantly, such activities may be separated using an endogenous secreted decoy receptor for RANKL.

### Discussion

The feasibility of OPG in combination with MC-GAG for effecting osteogenic differentiation with concurrent osteoclastogenic inhibition was examined in this Example. Using an adenoviral vector, it was demonstrated that OPG expression did not affect the viability or proliferation of primary hMSCs and that expression could be detected at even 8 weeks following transduction. With respect to endogenous RANKL and OPG, MC-GAG demonstrated a significantly lower RANKL/OPG protein expression ratio compared to Col-GAG. In the presence of AdOPG, the RANKL/OPG protein expression ratios were lowered even more so than baseline. Osteogenic differentiation in control versus AdOPG-transduced hMSCs did not demonstrate significant differences in terms of expression of osteogenic genes, phosphorylation of Smad1/5, or quantitative matrix mineralization in the absence of osteoclasts. In the presence of differentiating primary human osteoclasts in indirect co-cultures, mineralization was increased beyond hMSC single cultures, particularly in hMSCs cultured on MC-GAG. The augmentation of mineralization by hOCs persisted even with AdOPG transduction in indirect co-cultures, suggestive that paracrine effects were responsible for the positive regulation. Upon direct contact of differentiating osteoclasts with Col-GAG or MC-GAG materials a decrease in activities occurred which is partially rescued with the addition of differentiating control hMSCs. In the same direct co-cultures with AdOPG-transduced hMSCs, the resorptive activities of osteoclasts are completely abrogated. Surprisingly, the decrease in hOC resorptive activity by AdOPG resulted in an increase in activation of intracellular osteogenic mediators on both Col-GAG and MC-GAG. These results suggest several conclusions: 1) hMSC osteogenic differentiation on MC-GAG is largely unaffected by the addition of OPG; 2) osteoclast and hMSCs co-cultures positively regulate the activities of each other; 3) the direct osteogenic and osteoclastogenic coupling may be separated for the purposes of augmenting bone regeneration; 4) MC-GAG combined with OPG augments inhibition of osteoclast activity in the presence of differentiating hMSCs. This Example also demonstrates that the expression of OPG does not diminish osteoclast-induced mineralization suggesting that separate processes within the osteoclast control the paracrine stimulation of osteoprogenitors versus the resorptive activity of the osteoclast.

There was a clear difference in the RANKL/OPG ratio between hMSCs cultured in Col-GAG versus MC-GAG for either gene or protein expression (FIG. 2E and FIG. 2F). In protein expression, a statistically significant decrease in the RANKL/OPG ratio was evident on MC-GAG compared to Col-GAG which is consistent with the functional result of modestly diminishing resorptive capacity of the osteoclasts in the control hMSCs. However, there is a discrepancy between the protein versus gene expression ratios which MC-GAG demonstrating a higher RANKL/OPG ratio in transcription. In either material, the addition of AdOPG clearly diminishes the RANKL/OPG ratio in a significant manner. Furthermore, the addition of OPG predictably decreased osteoclast activity without significantly affecting the osteoblast lineage.

In the WST-1 analysis (FIG. 5A), the effect of osteoclast co-cultures with either empty, cell-free Col-GAG or MC-GAG is an increase in viability and proliferation. Additionally, there was seen an increased inhibition of osteoclast resorptive activity imparted by AdOPG-transduced cells on MC-GAG compared to Col-GAG (FIG. 5C). Although an adenoviral vector was utilized for OPG expression due to its high efficiency and high protein production, a high level of OPG protein was detected as late as eight weeks following transduction.

Despite the bidirectional positive regulation of osteogenic and osteoclastogenic differentiation in co-culture, the net effects of the direct co-cultures were tipped towards resorption in presence of endogenous hMSCs (FIGS. 7A and 7B), suggesting that the endogenous levels of OPG cannot overcome the osteoclast activation present in co-cultures. In the presence of AdOPG, not only did mineralization improve, Smad1/5 phosphorylation and Runx2 protein expression both increased. As this increase was not detected in single cultures of hMSCs transduced with AdOPG, these effects are most likely to be attributable to hOC-induced osteogenic effects. Similar to the paracrine effects on mineralization, co-cultures of AdOPG-transduced hMSCs on MC-GAG with hOCs may diminish hOC mediated resorptive capacities but do not mitigate the osteoinductive signals from hOCs.

### Example 2: Nanoparticulate Mineralized Collagen Glycosaminoglycan Materials Directly and Indirectly Inhibit Osteoclastogenesis and Osteoclast Activation

The ability of the extracellular matrix (ECM) to direct cell fate has generated the potential for developing a materials-only strategy for tissue regeneration. A nanoparticulate mineralized collagen glycosaminoglycan (MC-GAG) material that efficiently induced osteogenic differentiation of human mesenchymal stem cells (hMSCs) and calvarial bone healing without exogenous growth factors or progenitor cell expansion is described above. In this Example, interactions between MC-GAG and primary human osteoclasts (hOCs) were evaluated. In the absence of hMSCs, mineralized Col-GAG materials directly inhibited hOC viability, proliferation, and resorption in contrast to non-mineralized Col-GAG which demonstrated a modest inhibition of resorptive activity only. Co-cultures containing differentiating hMSCs with hOCs demonstrated increased hOC-mediated resorption only on Col-GAG while MC-GAG co-cultures continued to inhibit resorption. Unlike Col-GAG, hMSCs on MC-GAG expressed increased amounts of osteoprotegerin (OPG) protein, the major endogenous osteoclast inhibitor.

Interestingly, OPG expression was found to be antagonized by small mothers against decapentaplegicl/5 (Smad1/5) phosphorylation, an obligate pathway for osteogenic differentiation of hMSCs on MC-GAG, and potentiated by extracellular signal-regulated kinase (ERK1/2) phosphorylation. Collectively, these results suggested that the MC-GAG material both directly inhibited the osteoclast viability, proliferation, and resorptive activity as well as induced hMSCs to secrete osteoprotegerin, an anti-osteoclastogenic factor, via a signaling pathway distinct from osteogenic differentiation.

Fabrication and chemical crosslinking of non-mineralized and mineralized collagen scaffolds: Microfibrillar, type I collagen (Collagen Matrix, Oakland, N.J.) and chondroitin-6-sulfate (Sigma-Aldrich, St. Louis, Mo.) were combined in suspension in the absence and presence of calcium salts (calcium nitrate hydrate: Ca(NO₃)₂.4H₂O; calcium hydroxide: Ca(OH)₂, Sigma-Aldrich, St. Louis, Mo.) in an acetic acid (Col-GAG) or phosphoric acid (MC-GAG) solution. Using a constant cooling rate technique at a rate of 1° C./min, the solution was frozen from room temperature to −10° C. using a freeze dryer (Genesis, VirTis). Following sublimation of the ice phase, scaffolds were sterilized via ethylene oxide and cut into 8 mm disks for culture.

Crosslinking of scaffolds was performed after rehydration in phosphate buffered saline (PBS) for 4 hours using 1-ethyl-3-(3-dimethylaminopropyl) carbodiimide (EDAC, Sigma-Aldrich) and N-hydroxysuccinimide (NHS, Sigma Aldrich) at a molar ratio of 5:2:1 EDC:NHS:COOH where COOH represents the amount of collagen in the scaffold. Scaffolds were washed with PBS to remove any of the residual chemical.

Cell culture: Primary human mesenchymal stem cells (hMSCs; Lonza, Inc., Allendale, N.J.) were expanded in proliferation media composed of Dulbecco's Modified Eagle Medium (DMEM; Corning Cellgro, Manassas, Vt.) supplemented with 10% fetal bovine serum (FBS) (Atlanta Biologicals, Atlanta, Ga.), 2 mM L-glutamine (Life Technologies, Carlsbad, Calif.), 100 IU/mL penicillin/100 μg/mL streptomycin (Life Technologies).

Osteogenic differentiation of hMSCs on Col-GAG and MC-GAG: 3×10⁵ hMSCs were seeded onto 8 mm discs of CG-GAG and MC-GAG scaffolds in proliferation media. 24 h after seeding, media was switched to osteogenic differentiation media consisting of 10 mM β-glycerophosphate, 50 μg/mL ascorbic acid, and 0.1 μM dexamethasone. For inhibitor studies, scaffolds were treated or untreated with dorsomorphin homologue 1 (DMH1; Sigma-Aldrich) or PD98059 (Cell Signaling Technologies, Beverly, Mass.) separately, all at a concentration of 50 μM. Fresh DMH1 and PD98059 were added to each media change every 3 days.

Indirect hMSC and hOC co-cultures: 2×10⁵ hMSCs were seeded onto 6 mm Col-GAG and MC-GAG scaffolds in proliferation media. 24 h after seeding hMSCs, 6×10⁴ primary human osteoclast precursors (hOCs; Lonza, Inc., Allendale, N.J.) were separately cultured in Osteoclast Precursor Basal Medium (Lonza, Allendale N.J.) supplemented with 33 ng/mL macrophage-colony stimulating factor (M-CSF), 66 ng/mL of RANKL, 10 mM glycerophosphate, 50 μg/mL ascorbic acid, 0.1 μM dexamethasone on 24 well Corning Osteo Assay Surface Microplates (Corning, N.Y.), as the lower chamber of the co-culture. After 2 h, Col-GAG and MC-GAG scaffolds were transferred to 8 μm Transwell inserts (Corning, N.Y.), the upper chamber of the co-culture. Media were changed every 3 days for 3 weeks.

Direct hMSC and hOC co-cultures: 3.5×10⁵ hMSCs were seeded onto 8 mm Col-GAG and MC-GAG scaffolds in proliferation media for 24 h. 6×10⁴ hOCs were cultured in Osteoclast Precursor Basal Medium (Lonza, Allendale N.J.) supplemented with 33 ng/mL M-CSF, 66 ng/mL RANKL, 10 mM β-glycerophosphate, 50 μg/mL ascorbic acid, 0.1 dexamethasone on 24 well Osteo Assay Microplates. 2 h after hOCs were seeded, ColGAG and MC-GAG scaffolds were transferred to the Osteo Assay Plates and directly co-cultured with hOCs. Media were changed every 3 days for 2 weeks.

OPG Enzyme Linked Immunosorbent Assay (ELISA): Supernatants were collected from hMSC only, osteoclast only, or hMSC and hOC co-cultures. OPG protein concentrations were determined using the Human OPG DuoSet ELISA kit (R&D Systems, Minneapolis, Minn.) according to manufacturer's instructions. A 96 well microplate was coated with the capture antibody and incubated overnight at room temperature. After blocking, samples were incubated for 2 h at room temperature with the detection antibody followed by incubation with streptavidin-horseradish peroxidase (HRP) for 20 min. The reaction was quenched by adding 100 μl of 2N H₂SO₄. Plates were read at 450 and 540 nm wavelengths on the Epoch microplate reader (BioTex, Winooski, Vt.).

Microcomputed tomographic (micro-CT) imaging: Scaffolds were fixed using 10% formalin and mineralization was quantified by micro-computed tomographic imaging (micro-CT) using Scanco 35 (Scanco Medical AG, Bruttisellen, Switzerland) in triplicate for each timepoint. Scans were performed at medium resolution with a source voltage of 70 E (kVp) and I (μA) of 114. The images had a final element size of 12.5 μm. Images were analyzed using software supplied from Scanco (Image Processing Language version 5.6) and reconstructed into three-dimensional (3D) volumes of interest. Optimum arbitrary threshold values of 20 (containing scaffold and mineralization) and 80 (containing mineralization alone) were used uniformly for all specimens to quantify mineralized areas from surrounding unmineralized scaffold. Analysis of 3D reconstructions was performed using Scanco Evaluation script #2 (3D segmentation of two volumes of interest: solid dense in transparent low-density object) and script #6 (bone volume/density only bone evaluation) for volume determinations.

Western blot: Lysates were prepared from scaffolds at 0, 4, 14, and 24 days of culture using SDS sample buffer and equal amounts were subjected to 4-20% SDS-PAGE (Bio-Rad, Hercules, Calif.). Western blot analysis was carried out with antibodies against OPG, RANKL, phosphorylated small mothers against decapentaplegic 1/5 (p-Smad1/5), total Smad5, phosphorylated extracellular regulated kinase 1/2 (p-ERK1/2), total ERK1/2, and (3-actin followed by 1:4000 dilutions of HRP-conjugated IgG antibodies (Bio-Rad, Hercules, Calif.) and an enhanced chemiluminescent substrate (Thermo Scientific, Rockford, Ill.). For detection of p-Smad1/5 and total Smad5, 10 μg of lysate was loaded per lane. For detection of OPG, RANKL, p-ERK1/2, total ERK1/2, and β-actin, 20 μg of lysate was loaded per lane. All antibodies were obtained from Cell Signaling Technologies (Beverly, Mass.) with the exception of RANKL, OPG and β-actin antibodies which were obtained from Santa Cruz Biotechnology (Santa Cruz, Calif.). Imaging analysis was carried out using ImageJ (NIH, Bethesda, Md.).

Water Soluble Tetrazolium-1 (WST-1) Assay: Culture media was supplemented with cell proliferation reagent WST-1 (Roche, Basel, Switzerland) at a 1:10 concentration. Scaffolds were incubated for 3-4 h at 37° C. in a humidified atmosphere with 5% CO₂. Absorbance of the incubation medium was measured at 450 and 690 nm (Epoch spectrophotometer, BioTek, Winooski, Vt.).

Tartrate-Resistant Acid Phosphatase (TRAP) Staining: hOCs were detected using Leukocyte TRAP Kit 387-A (Sigma-Aldrich) according to the manufacturer's instructions. Cultured cells were fixed with formaldehyde for 5 min at room temperature, washed, and air-dried. After staining, TRAP-positive multinucleated cells were observed under a phase-contrast microscope at 20× magnification and digitally photographed.

Resorption Pit Assay: Activity of hOCs in single culture or co-cultured with scaffolds with and without hMSCs was evaluated for resorption pit formation on Osteo Assay microplates. At the completion of the culture period, culture media was aspirated and 500 μL of 10% bleach solution was added for 5 minutes at room temperature. The wells were washed with distilled water and allowed to dry at room temperature for 3-5 h. Pits were observed using a standard microscope and digitally photographed. Percentage of resorption for the whole well of the culture at magnification 2× was calculated by ImageJ.

Statistical analysis: All statistical analyses were performed using SPSS Version 24 (Chicago, Ill.). Data points were composed of duplicates of at least three independent experiments, unless otherwise indicated. Mean measurements of mRNA expression were analyzed for statistical significance by analyses of variance (ANOVA) followed by post hoc tests using the Tukey criterion. A value of p<0.05 was considered significant.

Results

hMSCs Undergoing Osteogenic Differentiation Induce Expression of Osteoprotegerin in a Differential Manner on Non-Mineralized Versus Mineralized Collagen Glycosaminoglycan Materials

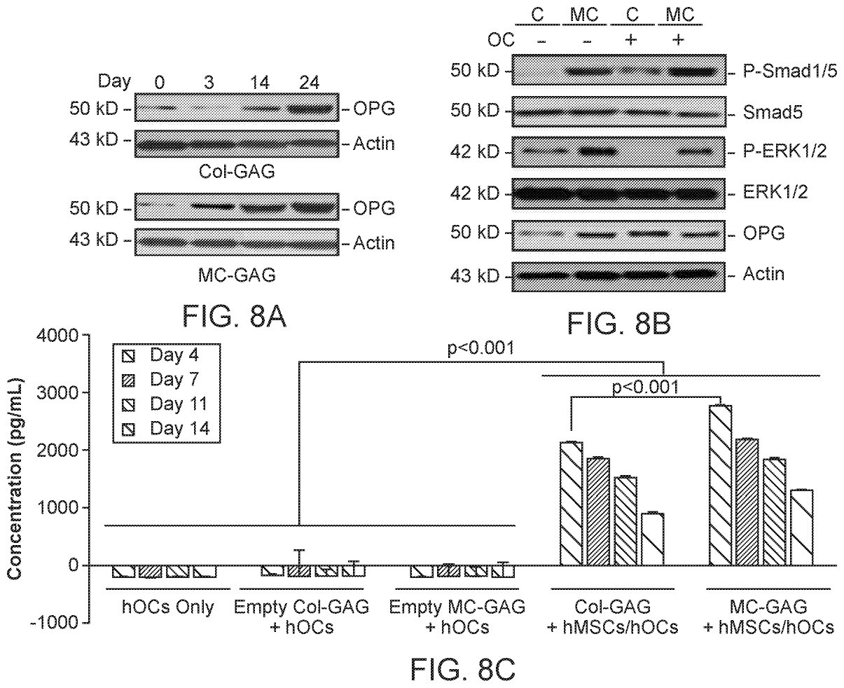

As previously discussed MC-GAG is capable of inducing in vitro hMSC osteogenic differentiation and mineralization as well as in vivo bone healing beyond that of a non-mineralized Col-GAG control material. To evaluate the role of MC-GAG in the regulation of osteoclast activation during osteoprogenitor differentiation, bone marrow-derived primary hMSCs (CD105+CD166+CD29+CD44+CD14-CD34-CD45-) were cultured in osteogenic differentiation medium and expression of OPG protein was evaluated (FIGS. 8A and 8B). Over the course of osteogenic differentiation, OPG protein expression was significantly increased in MC-GAG at day 3, 14, and 24 when compared to hMSCs differentiated on Col-GAG as assessed by western blot analysis (FIG. 8A).

To understand the coordination of osteoclast differentiation and hMSCs undergoing osteogenic differentiation on Col-GAG and MC-GAG, an indirect in vitro co-culture assay was devised. hMSCs seeded on Col-GAG and MC-GAG were cultured in the upper chamber of Transwell inserts in the absence or presence of primary hOCs plated in the lower chamber. Co-cultures were induced to simultaneously undergo osteogenic and osteoclastogenic differentiation with RANKL (66 ng/mL), M-CSF (33 ng/mL), □-glycerophosphate, and dexamethasone for three weeks and western blot analysis of the cultures were performed. In the presence of osteoclasts, the expression of phosphorylated Smad1/5 (p-Smad1/5) increased significantly in both Col-GAG and MC-GAG scaffolds (FIG. 8B). Additionally, the expression of OPG also increased for hMSCs on Col-GAG in the presence of hOCs but not on MC-GAG in the presence of hOCs while ERK1/2 phosphorylation was decreased in hOC co-cultures.

To determine the effect of hOCs on OPG secretion, OPG ELISAs were performed (FIG. 8C). Significant differences in secreted OPG were found between cultures on ANOVA [F(15,48)=172.56, p<0.001]. OPG was undetected in the absence of hMSCs (hOC Only, Empty Col-GAG/hOCs, or Empty MC-GAG/hOCs) when compared to co-cultures with hMSCs at every timepoint on posthoc comparisons (p<0.001). Between co-cultures of hOCs with differentiating hMSCs on Col-GAG versus MC-GAG, OPG was elevated in MC-GAG co-cultures particularly at day 4 (p<0.001). At later timepoints, OPG trended higher in MC-GAG co-cultures, but was no longer found to be significant.

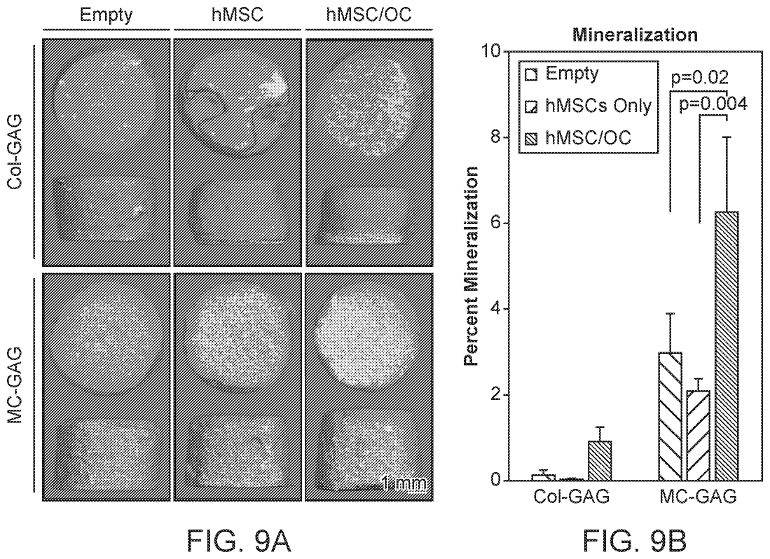

hOCs augment hMSC mineralization on MC-GAG To understand the effects of primary human osteoclast differentiation on hMSCs on Col-GAG and MC-GAG, mineralization in hMSCs undergoing osteogenic differentiation in the absence and presence of hOCs was evaluated (FIGS. 9A and 9B). Empty scaffolds, scaffolds seeded with hMSCs, or co-cultures with hMSCs on scaffolds in Transwell inserts and hOCs in the lower chamber were evaluated with micro-CT scanning after 3 weeks of culture in concurrent osteogenic and osteoclastogenic differentiation medium. Overall, differences in mineralization were found to be present [F(5,8)=22.44, p<0.001]. Posthoc comparisons between groups did not demonstrate statistically significant differences between mineralization on empty scaffolds or scaffolds cultured with hMSCs as single cultures on either material, an expected result at 3 weeks of culture. However, in the presence of hOCs, a significant increase in new mineral formation was seen in MC-GAG when compared to empty scaffolds (p=0.02) or scaffolds cultured with hMSCs in single culture (p=0.004). While co-cultures of hOC and hMSCs cultured on Col-GAG demonstrated a qualitative increase in mineralization, this difference did not reach statistical significance. These data suggest that hOCs increase hMSC osteogenic differentiation on MC-GAG scaffolds via soluble factors in a paracrine fashion.

MC-GAG Diminishes hOC Activation and Resorption Directly and Indirectly

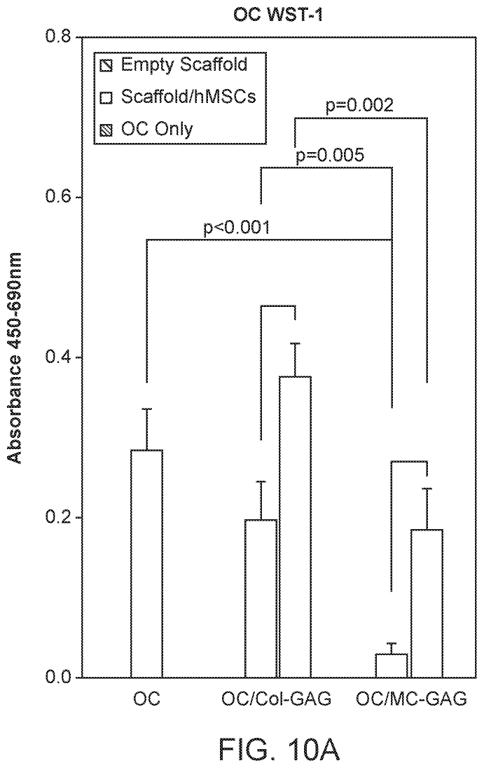

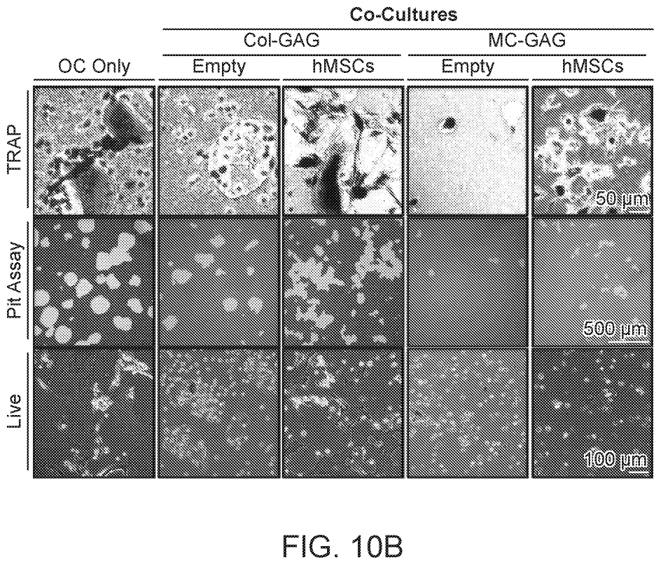

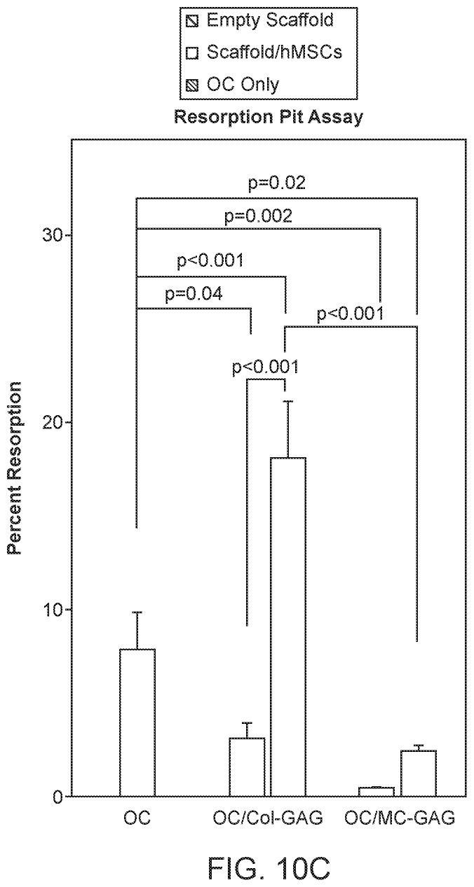

Osteoclasts were evaluated in a direct co-culture system in order to account for both direct and indirect effects on osteoclast activity (FIGS. 10A-10C). hMSCs were cultured on Col-GAG or MC-GAG materials that were then co-cultured directly with primary hOCs 24 hours after seeding. The co-cultures were differentiated simultaneously in osteogenic differentiation medium supplemented with M-CSF (33 ng/mL) and RANKL (33 ng/mL). At 14 days of culture, hOC proliferation and viability on the plate were assessed with WST-1 assays and found to demonstrate significant differences between the conditions by ANOVA [F(4,10)=26.38, p<0.001] (FIG. 10A). Post hoc comparisons between groups demonstrated that hOC viability and proliferation were significantly diminished in the presence of empty MC-GAG (p<0.001) but not Col-GAG scaffolds when compared to hOCs alone. When comparing the differences between empty versus hMSC-seeded materials, osteogenic differentiation of hMSCs clearly demonstrated an increase in hOC viability and/or proliferation on both Col-GAG (p=0.004) and MC-GAG (p=0.009) materials. However, the increase in hOC viability and proliferation in the presence of hMSCs on MC-GAG was significantly lower compared to hMSCs on Col-GAG (p=0.002).

To evaluate hOC differentiation and activity, TRAP staining and resorption pit assays were performed for each co-culture condition and corresponding controls (FIG. 10B). Both TRAP staining and resorption were diminished in co-culture with either empty Col-GAG or MC-GAG. Additionally, live images demonstrated qualitatively small rounded cells as opposed to large, differentiated multi-nucleated osteoclasts. When co-cultured with differentiating hMSCs on Col-GAG, TRAP staining and resorption pits increased. Simultaneously, an increase in larger, multi-nucleated cells was clearly evident in live cell imaging. When co-cultured with differentiating hMSCs on MC-GAG, TRAP staining and resorption pits increased, however this was qualitatively less compared to hOC single culture or hOCs co-cultured with hMSCs on Col-GAG.

To objectively evaluate the differences in hOC activity, percentages of the resorption were quantified for the different conditions (FIG. 10C). Significant differences were found between the conditions on ANOVA [F(4,10)=53.98, p<0.001]. On posthoc comparisons, both co-culture conditions of hOC with empty Col-GAG or MC-GAG materials demonstrated decreased resorptive activity (p=0.04 and p=0.002, respectively). Similar to the WST-1 results, addition of hMSCs to the materials resulted in augmentation of resorptive activity. In the presence of hMSCs differentiated on Col-GAG, hOC-mediated resorption was significantly higher than in the presence of empty Col-GAG (p<0.001) as well as hOC single cultures (p<0.001). In the presence of hMSCs differentiated on MC-GAG, the recovery of hOC-mediated resorption was significantly less compared to that mediated by hMSCs differentiated on Col-GAG (p<0.001) or hOC single cultures (p=0.02).

OPG Expression on MC-GAG is Upregulated by ERK1/2 Activation and Antagonized by Canonical BMPR Signaling

There is differential regulation of osteogenic differentiation and mineralization of primary hMSCs on Col-GAG and MC-GAG. MC-GAG demonstrated an autogenous activation of the BMPR signaling in hMSCs that greatly surpasses Col-GAG. In both materials, BMPR signaling was essential for mineralization, however, Col-GAG also requires MEK1/ERK1/2-mediated signaling for mineralization whereas MC-GAG-mediated mineralization was completely independent of ERK1/2 phosphorylation.

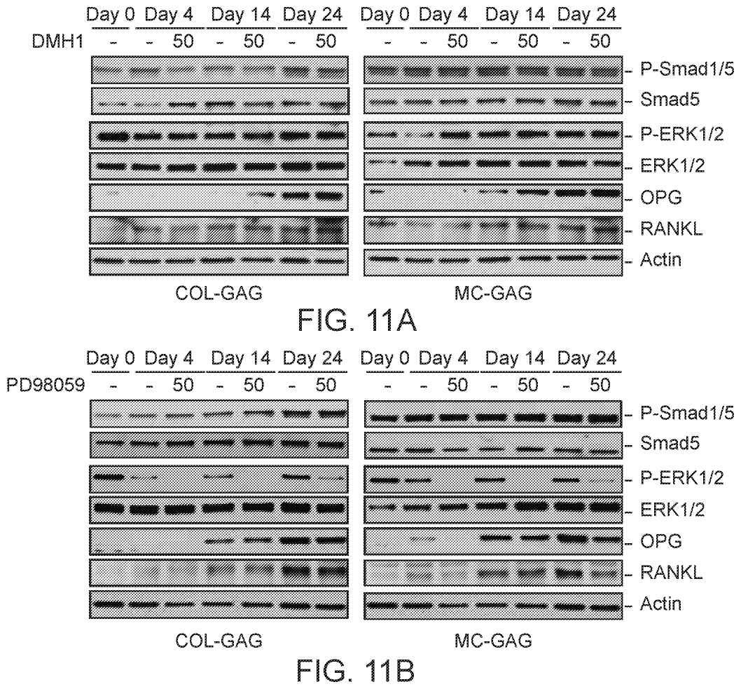

To characterize the mechanism responsible for differential OPG expression osteogenically differentiated hMSCs cultured on MC-GAG versus Col-GAG, DMH1 and PD98059 small molecule inhibitors were utilized for the canonical BMP receptor and MEK1/ERK1/2 signaling pathways, respectively (FIG. 11). In the presence of DMH1, phosphorylation of Smad1/5 was partially decreased at all timepoints in either material on western blot analyses (FIGS. 11A-11B). Due to the massive upregulation of Smad1/5 phosphorylation in MC-GAG, a smaller inhibition was noted compared to Col-GAG. No differences in total Smad5, total ERK1/2, RANKL, or actin were detected. In contrast, OPG expression was increased in the presence of DMH1 on both Col-GAG and MC-GAG, particularly at the day 14 timepoint. A small elevation in phosphorylated ERK1/2 could also be seen in the presence of DMH1 specifically in MC-GAG at day 4.

To determine the contribution of the ERK1/2-mediated pathways, primary hMSCs osteogenically differentiated on Col-GAG or MC-GAG were treated with PD98059 (FIG. 11B). As expected PD98059 decreased phosphorylated ERK1/2 at all timepoints in either material, whereas no effects were noted for phosphorylated Smad1/5, total Smad5, total ERK1/2, or actin. Unlike DMH1, OPG protein expression was decreased in MC-GAG in the presence of PD98059 at all timepoints, while no differences were detected on Col-GAG. Similarly, RANKL expression was also downregulated, particularly at day 24, in the presence of PD98059 on MC-GAG whereas no differences were detected on Col-GAG. In combination, these data suggest that the necessary signaling mechanisms for osteogenic differentiation are both distinct and antagonistic to the mechanisms responsible for osteoclast regulation in hMSCs differentiated on MC-GAG.

### Discussion

Applicants investigated the effect of nanoparticulate mineralized collagen glycosaminoglycan on osteoclastogenesis and osteoclast activity. Through activation of osteogenic differentiation, MC-GAG and Col-GAG, its non-mineralized counterpart, induced an elevation of OPG protein expression by osteoprogenitors (FIG. 8A). Early in differentiation, MC-GAG surpassed Col-GAG in the amount of total OPG protein and the amount of secreted OPG (FIG. 8C). The quantity of OPG protein expression was potentiated by the addition of differentiating osteoclasts in co-culture (FIG. 8B). With respect to differentiating hMSCs, the presence of hOCs on either material augmented the canonical BMPR signaling pathway as seen by an increase in phosphorylated Smad1/5 (FIG. 8) as well as mineralization particularly on MC-GAG (FIG. 9). When the interactions between materials and hOCs were investigated, hOCs cultured in the presence of either Col-GAG or MC-GAG demonstrated a significant decrease in osteoclast-mediated resorption in the absence of hMSCs (FIG. 10C). While Col-GAG only affected resorption but not viability or proliferation, MC-GAG appeared to diminish both osteoclast activity and viability/proliferation (FIGS. 10A and 10C). The addition of differentiating hMSCs on either material improved hOC viability/proliferation significantly (FIG. 10A).

While osteogenic differentiation of hMSCs on Col-GAG augmented osteoclast resorption (FIG. 10C), hOCs in the presence of MC-GAG continued to demonstrate diminished resorptive activity suggesting an indirect inhibitory effect of differentiating osteoprogenitors on MC-GAG. To mechanistically understand the indirect effects induced by MC-GAG on osteoclasts through osteogenic differentiation of hMSCs, the canonical BMPR and ERK1/2 pathways were inhibited with respective small molecule inhibitors (FIG. 11). Inhibition of the canonical BMPR pathway resulted in an increase in OPG protein expression by hMSCs cultured on both Col-GAG and MC-GAG materials. In contrast, inhibition of ERK1/2 phosphorylation downregulated both OPG and RANKL expression on MC-GAG whereas Col-GAG was not affected. These results suggest several conclusions regarding the direct and indirect influences of MC-GAG on hOC viability, proliferation, and activity: 1. Col-GAG and MC-GAG have direct inhibitory effects on osteoclast resorptive activity; 2. MC-GAG possesses an additional intrinsic ability to directly diminish osteoclast viability and proliferation that is not present in Col-GAG; 3. Indirectly, the addition of hMSCs undergoing osteogenic differentiation improves osteoclast viability or proliferation on either Col-GAG or MC-GAG; 4. Differentiating hMSCs on MC-GAG continue to inhibit the resorptive activity of hOCs whereas Col-GAG augments hOC-mediated resorption, correlating to the increased protein expression and secretion of OPG from hMSCs on MC-GAG. 5. While the canonical BMPR signaling pathway is essential for osteogenic differentiation of hMSCs cultured on either Col-GAG or MC-GAG, the MEK1/ERK1/2 pathway regulates OPG and RANKL expression on MC-GAG but not Col-GAG.

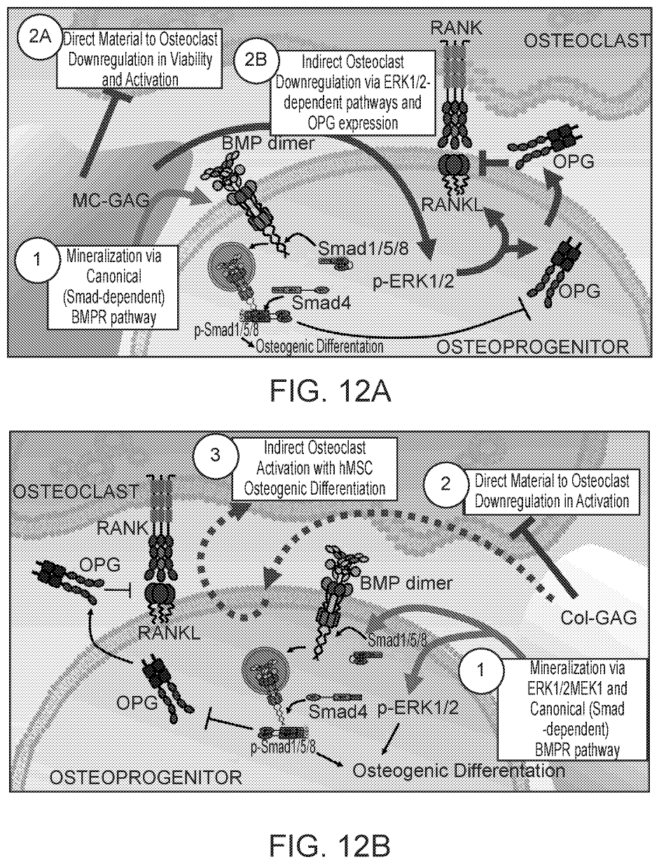

The combination of these conclusions suggests differing models in the interactions between Col-GAG and MC-GAG with osteoprogenitors and osteoclast progenitors (FIGS. 12A and 12B). With respect to osteoprogenitors, the necessary mechanism for osteogenic differentiation was an autogenous activation of the canonical BMP receptor signaling pathway through elevated Smad1/5 phosphorylation for both Col-GAG and MC-GAG (FIG. 12A, Mechanism 1; FIG. 12B, Mechanism 1). Whereas Col-GAG also required activation of the ERK1/2 pathway, MC-GAG was independent of ERK1/2 for mineralization. With respect to osteoclast progenitors, both Col-GAG and MC-GAG had the ability to directly diminish osteoclast activation and resorptive activity (FIG. 12A, Mechanism 2A; FIG. 12B, Mechanism 2), thus suggesting that the activation of osteoclasts is diminished in the presence of collagen and glycosaminoglycan in the form of chondroitin-6-sulfate.

Unlike Col-GAG, the direct inhibition of osteoclasts by MC-GAG materials is also accompanied with a diminished viability and proliferation of osteoclast precursors, suggesting a role for nanoparticulate mineral content in decreasing osteoclast activity and proliferation. In the presence of differentiating osteoprogenitors, differing net effects on osteoclast activation are observed between Col-GAG and MC-GAG (FIG. 12A, Mechanism 2B; FIG. 12B, Mechanism 3). Although both materials induce OPG expression by osteoprogenitors, the quantity of OPG expression is higher in hMSCs differentiated on MC-GAG which is correlated to a net effect of continued inhibition of osteoclast-mediated resorption (FIG. 12A, Mechanism 2B). While differentiating osteoprogenitors on either material improves the viability and/or proliferation of osteoclasts, a net effect of increased resorptive activity is only observed in the presence of Col-GAG (FIG. 12B, Mechanism 3).

Direct inhibitory effects of Col-GAG and MC-GAG on hOC-mediated resorption are likely to be related to glycosaminoglycan content. In terms of RANKL sequestration, the inhibitory effects on osteoclasts can be overcome with increases in RANKL concentration. Both Col-GAG and MC-GAG-mediated osteoclast inhibition require direct contact of the material with cells such that indirect cultures (via Transwell inserts) demonstrate no statistically significant effects on resorption.

MC-GAG also demonstrated an additional direct effect of reducing hOC viability and proliferation. As the major difference in composition between Col-GAG and MC-GAG is the presence of nanoparticulate calcium phosphate in the latter, one potential explanation would be negative regulation by calcium or phosphate ion-induced signaling pathways. High levels of extracellular calcium ion have been identified to induce osteoclast apoptosis dependent on L-type voltage gated calcium channels but not the calcium sensing receptor [51]. Similarly, high extracellular phosphate concentrations have also been identified to inhibit osteoclastogenesis as well as induce osteoprogenitors to upregulate osteoprotegerin, thereby acting as both a direct and indirect inhibitor of osteoclastogenesis and osteoclast activation [50]. Conversely, both calcium and phosphate ions are known to be activators of osteogenic differentiation [22], [49], and [52]. Thus, bone regenerative materials that include mineral content are likely to be able to utilize the dichotomy of osteogenic activation and osteoclast inhibition imparted by calcium and phosphate ion signaling.

A second inhibitory effect on osteoclast activity was also produced indirectly by osteoprogenitors differentiated on MC-GAG. The correlation of increased OPG expression by osteoprogenitors on MC-GAG suggests that the anti-resorptive effect may be due to an alteration in the relative equilibrium between RANKL and OPG within this system. The increase OPG secretion in MC-GAG was higher than Col-GAG only early in differentiation (day 4). Thus, further in vivo studies evaluating the effects of MC-GAG on local osteoclast differentiation may be necessary to understand the ultimate net effects of MC-GAG on OPG over time.

Although OPG is generally considered a marker of osteogenic differentiation, mechanisms governing OPG expression in osteoprogenitors on Col-GAG and MC-GAG are clearly distinct from the necessary pathways for mineralization. Whereas both Col-GAG and MC-GAG requires Smad1/5 phosphorylation for hMSC mineralization, the canonical BMPR pathway antagonized OPG expression such that inhibition by DMH1 resulted in higher levels of OPG. Both OPG and RANKL expression in hMSCs depended upon ERK1/2 phosphorylation for MC-GAG but not Col-GAG. These data suggested that osteogenic differentiation and expression of osteoclast regulatory proteins may be separately modulated by specific extracellular matrix compositions in regenerative materials.

### Example 3: Col-GAG or MC-GAG for Regeneration of Rabbit Calvarial Defects

Although AdOPG-transduced hMSCs offer a simple method of delivering OPG for proof of principle in vitro studies, the likelihood of delivering adenovirally infected hMSCs in the clinical scenario is not practical and, potentially, not necessary for several reasons: 1. Safety of adenoviral infection, 2. Requirement for osteoclasts for remodeling. When immunofluorescent histochemistry was performed on in vivo sections of Col-GAG or MC-GAG regenerated rabbit calvarial defects 12 weeks after implantation, anti-TRAP staining clearly detected osteoclasts in both types of regenerated bone without significant differences in quantity (FIG. 13). Additionally, this data suggests that cell-free MC-GAG is equally effective as BMSC-loaded MC-GAG for in vivo calvarial regeneration (FIG. 13). Thus, cell-free delivery of OPG on MC-GAG as a composite material may be a potentially useful for augmentation of calvarial regeneration.

To test this hypothesis, two types of MC-GAG/osteoprotegerin (MCGO), materials using an immersion technique were prepared as follows. The two types of scaffold are scaffolds incorporated with osteoprotegerin non-covalently (“CGO” for Col-GAG and “MCGO” for MC-GAG), and scaffolds incorporated with osteoprotegerin covalently (“CGOX” for Col-GAG and “MCGOX” for MC-GAG) Col-GAG and MC-GAG scaffolds were fabricated by lyophilizing a suspension of microfibrillar type I collagen (Collagen Matrix, Oakland, N.J.), chondroitin-6-sulfate in a solution of 0.05 M acetic acid (pH 3.2) or with calcium salts (calcium nitrate hydrate: Ca(NO₃)₂.4H₂O; calcium hydroxide: Ca(OH)₂, Sigma-Aldrich) in a solution of phosphoric acid, respectively.

Following freezing using a constant cooling rate technique (1° C./min) from room temperature to −10° C. using a freeze dryer (Genesis, VirTis), the ice phase was sublimated under vacuum (<200 mTorr, 0° C.) generating a scaffold porosity of 85±3%, pore size of 156±6 μm, and Ca:P ratio of 1:1 similar to brushite for MC-GAG. Scaffolds were then divided into two methods for OPG incorporation. For non-covalent incorporation of OPG, scaffolds were crosslinked in a solution of 1-ethyl-3-(3-dimethylaminopropyl) carbodiimide (EDC, Sigma Aldrich) and N-hydroxysuccinimide (NHS, Sigma Aldrich) at a molar ratio of 5:2:1 EDC:NHS:COOH where COOH represents the amount of collagen in the scaffold. Subsequently, scaffolds were immersed in 50 μg/mL of purified OPG (Peprotech, Rocky Hill, N.J.) in PBS overnight at 4° C. to generate CGO and MCGO. For covalent incorporation of OPG, crosslinking of scaffolds were performed with 50 μg/mL of OPG (Peprotech) to generate CGOX and MCGOX. All four types of scaffolds were washed in PBS following fabrication.

Alternatively, scaffolds with non-covalently incorporated OPG (or OPG fragment) may be prepared by lyophilizing a suspension of microfibrillar type I collagen (Collagen Matrix, Oakland, N.J.), chondroitin-6-sulfate in a solution of 0.05 M acetic acid (pH 3.2) and OPG (or an OPG fragment) to prepare scaffolds.

Scaffolds with covalently incorporated OPG may be prepared by lyophilizing a suspension of microfibrillar type I collagen (Collagen Matrix, Oakland, N.J.), chondroitin-6-sulfate in a solution of calcium salts (calcium nitrate hydrate: Ca(NO₃)₂.4H₂O; calcium hydroxide: Ca(OH)₂, Sigma-Aldrich), phosphoric acid and OPG, (or OPG fragment).

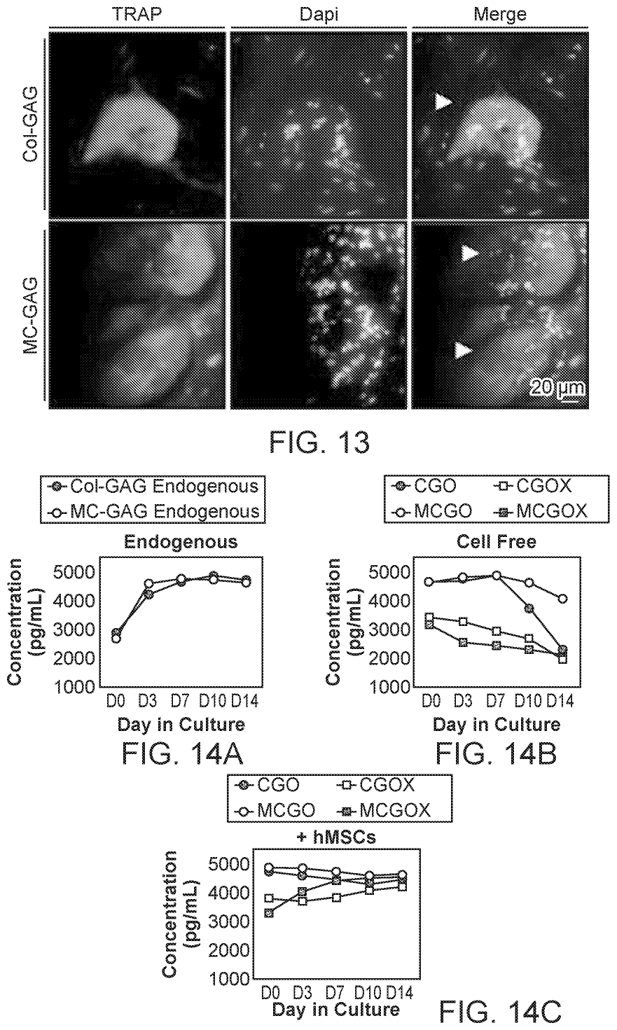

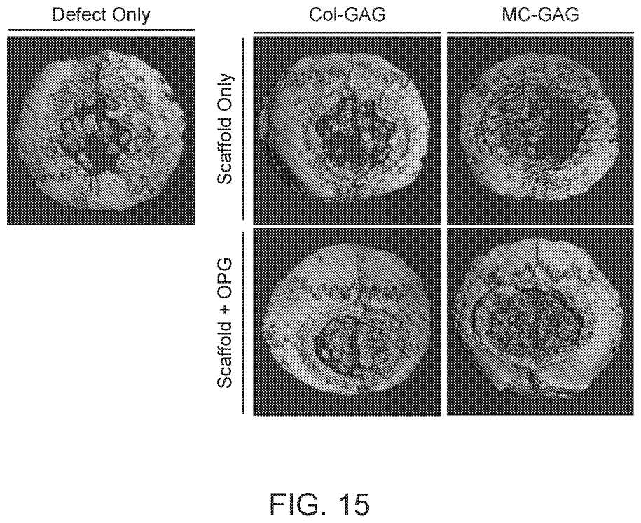

When hMSCs were differentiated on Col-GAG or MC-GAG in the absence of exogenous OPG, soluble endogenous OPG was detected on enzyme linked immunosorbent assays (ELISAs) in a manner increasing from day 0 to day 7 of culture before tapering (FIG. 14A). When OPG composite scaffolds were evaluated in a cell-free manner, both non-covalently incorporated (CGO and MCGO) and covalently incorporated (CGOX and MCGOX) demonstrated elution (FIG. 14B). Whereas CGO and MCGO exhibited the highest amounts of elution between 0-7 days with a tapering beyond day 7, CGOX and MCGOX eluted in a lower and slower amount. When hMSCs were differentiated on the respective OPG scaffolds, CGO and MCGO both showed a consistent level of soluble OPG from day 0-14 of culture at approximately the maximum concentrations achieved in both endogenous and exogenous circumstances. Similar to cell-free conditions, CGOX and MCGOX started at a level higher than that of endogenous but lower than that of the non-covalent scaffolds with a slight increase over time. Taken together, this data establishes two methods of OPG incorporation on collagen glycosaminoglycan scaffolds which allow for differences in temporal release of OPG. It is believed that OPG composite scaffolds will allow for improved in vivo calvarial bone healing via osteoclast inhibitory effects.

### EQUIVALENTS

It should be understood that although the present invention has been specifically disclosed by certain aspects, embodiments, refinements and optional features, modification, improvement and variation of such aspects, embodiments, and optional features can be resorted to by those skilled in the art, and that such modifications, improvements and variations are considered to be within the scope of this disclosure. All referenced cited herein are incorporated by reference.

The invention has been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

### REFERENCES

[1] A. Leibbrandt, J. M. Penninger, Novel functions of RANK(L) signaling in the immune system, Adv Exp Med Biol 658 (2010) 77-94.
[2] K. Fuller, B. Wong, S. Fox, Y. Choi, T. J. Chambers, TRANCE is necessary and sufficient for osteoblast-mediated activation of bone resorption in osteoclasts, J Exp Med 188(5) (1998) 997-1001.
[3] D. L. Lacey, E. Timms, H. L. Tan, M. J. Kelley, C. R. Dunstan, T. Burgess, R. Elliott, A. Colombero, G. Elliott, S. Scully, H. Hsu, J. Sullivan, N. Hawkins, E. Davy, C. Capparelli, A. Eli, Y. X. Qian, S. Kaufman, I. Sarosi, V. Shalhoub, G. Senaldi, J. Guo, J. Delaney, W. J. Boyle, Osteoprotegerin ligand is a cytokine that regulates osteoclast differentiation and activation, Cell 93(2) (1998) 165-76.
[4] H. Yasuda, N. Shima, N. Nakagawa, K. Yamaguchi, M. Kinosaki, S. Mochizuki, A. Tomoyasu, K. Yano, M. Goto, A. Murakami, E. Tsuda, T. Morinaga, K. Higashio, N. Udagawa, N. Takahashi, T. Suda, Osteoclast differentiation factor is a ligand for osteoprotegerin/osteoclastogenesis-inhibitory factor and is identical to TRANCE/RANKL, Proc Natl Acad Sci USA 95(7) (1998) 3597-602.
[5] N. Nakagawa, M. Kinosaki, K. Yamaguchi, N. Shima, H. Yasuda, K. Yano, T. Morinaga, K. Higashio, RANK is the essential signaling receptor for osteoclast differentiation factor in osteoclastogenesis, Biochem Biophys Res Commun 253(2) (1998) 395-400.
[6] J. C. Lee, L. Spiguel, D. S. Shenaq, M. Zhong, C. Wietholt, T. C. He, R. R. Reid, Role of RANK-RANKL-OPG axis in cranial suture homeostasis, J Craniofac Surg 22(2) (2011) 699-705.
[7] J. B. Maxhimer, J. P. Bradley, J. C. Lee, Signaling pathways in osteogenesis and osteoclastogenesis: Lessons from cranial sutures and applications to regenerative medicine, Genes Dis 2(1) (2015) 57-68.
[8] D. M. Anderson, E. Maraskovsky, W. L. Billingsley, W. C. Dougall, M. E. Tometsko, E. R. Roux, M. C. Teepe, R. F. DuBose, D. Cosman, L. Galibert, A homologue of the TNF receptor and its ligand enhance T-cell growth and dendritic-cell function, Nature 390(6656) (1997) 175-9.
[9] Y. Y. Kong, H. Yoshida, I. Sarosi, H. L. Tan, E. Timms, C. Capparelli, S. Morony, A. J. Oliveira-dos-Santos, G. Van, A. Itie, W. Khoo, A. Wakeham, C. R. Dunstan, D. L. Lacey, T. W. Mak, W. J. Boyle, J. M. Penninger, OPGL is a key regulator of osteoclastogenesis, lymphocyte development and lymph-node organogenesis, Nature 397(6717) (1999) 315-23.
[10] J. Li, I. Sarosi, X. Q. Yan, S. Morony, C. Capparelli, H. L. Tan, S. McCabe, R. Elliott, S. Scully, G. Van, S. Kaufman, S. C. Juan, Y. Sun, J. Tarpley, L. Martin, K. Christensen, J. McCabe, P. Kostenuik, H. Hsu, F. Fletcher, C. R. Dunstan, D. L. Lacey, W. J. Boyle, RANK is the intrinsic hematopoietic cell surface receptor that controls osteoclastogenesis and regulation of bone mass and calcium metabolism, Proc Natl Acad Sci USA 97(4) (2000) 1566-71.
[11] T. J. Yun, M. D. Tallquist, A. Aicher, K. L. Rafferty, A. J. Marshall, J. J. Moon, M. E. Ewings, M. Mohaupt, S. W. Herring, E. A. Clark, Osteoprotegerin, a crucial regulator of bone metabolism, also regulates B cell development and function, J Immunol 166(3) (2001) 1482-91.
[12] W. S. Simonet, D. L. Lacey, C. R. Dunstan, M. Kelley, M. S. Chang, R. Lüthy, H. Q. Nguyen, S. Wooden, L. Bennett, T. Boone, G. Shimamoto, M. DeRose, R. Elliott, A. Colombero, H. L. Tan, G. Trail, J. Sullivan, E. Davy, N. Bucay, L. Renshaw-Gegg, T. M. Hughes, D. Hill, W. Pattison, P. Campbell, S. Sander, G. Van, J. Tarpley, P. Derby, R. Lee, W. J. Boyle, Osteoprotegerin: a novel secreted protein involved in the regulation of bone density, Cell 89(2) (1997) 309-19.
[13] L. M. Flick, J. M. Weaver, M. Ulrich-Vinther, F. Abuzzahab, X. Zhang, W. C. Dougall, D. Anderson, R. J. O'Keefe, E. M. Schwarz, Effects of receptor activator of NFkappaB (RANK) signaling blockade on fracture healing, J Orthop Res 21(4) (2003) 676-84.
[14] A. D. Barrow, N. Raynal, T. L. Andersen, D. A. Slatter, D. Bihan, N. Pugh, M. Cella, T. Kim, J. Rho, T. Negishi-Koga, J. M. Delaisse, H. Takayanagi, J. Lorenzo, M. Colonna, R. W. Farndale, Y. Choi, J. Trowsdale, OSCAR is a collagen receptor that costimulates osteoclastogenesis in DAP12-deficient humans and mice, J Clin Invest 121(9) (2011) 3505-16.
[15] S. Herman, R. B. Müller, G. Krönke, J. Zwerina, K. Redlich, A. J. Hueber, H. Gelse, E. Neumann, U. Müller-Ladner, G. Schett, Induction of osteoclast-associated receptor, a key osteoclast costimulation molecule, in rheumatoid arthritis, Arthritis Rheum 58(10) (2008) 3041-50.
[16] J. C. Lee, E. J. Volpicelli, Bioinspired Collagen Scaffolds in Cranial Bone Regeneration: From Bedside to Bench, Adv Healthc Mater (2017).
[17] J. Salbach-Hirsch, N. Ziegler, S. Thiele, S. Moeller, M. Schnabelrauch, V. Hintze, D. Scharnweber, M. Rauner, L. C. Hofbauer, Sulfated glycosaminoglycans support osteoblast functions and concurrently suppress osteoclasts, J Cell Biochem 115(6) (2014) 1101-11.
[18] J. Salbach, S. Kliemt, M. Rauner, T. D. Rachner, C. Goettsch, S. Kalkhof, M. von Bergen, S. Möller, M. Schnabelrauch, V. Hintze, D. Scharnweber, L. C. Hofbauer, The effect of the degree of sulfation of glycosaminoglycans on osteoclast function and signaling pathways, Biomaterials 33(33) (2012) 8418-29.
[19] J. Salbach-Hirsch, J. Kraemer, M. Rauner, S. A. Samsonov, M. T. Pisabarro, S. Moeller, M. Schnabelrauch, D. Scharnweber, L. C. Hofbauer, V. Hintze, The promotion of osteoclastogenesis by sulfated hyaluronan through interference with osteoprotegerin and receptor activator of NF-κB ligand/osteoprotegerin complex formation, Biomaterials 34(31) (2013) 7653-61.
[20] A. Miyauchi, K. A. Hruska, E. M. Greenfield, R. Duncan, J. Alvarez, R. Barattolo, S. Colucci, A. Zambonin-Zallone, S. L. Teitelbaum, A. Teti, Osteoclast cytosolic calcium, regulated by voltage-gated calcium channels and extracellular calcium, controls podosome assembly and bone resorption, J Cell Biol 111(6 Pt 1) (1990) 2543-52.
[21] A. Mozar, N. Haren, M. Chasseraud, L. Louvet, C. Maziere, A. Wattel, R. Mentaverri, P. Morlière, S. Kamel, M. Brazier, J. C. Maziere, Z. A. Massy, High extracellular inorganic phosphate concentration inhibits RANK-RANKL signaling in osteoclast-like cells, J Cell Physiol 215(1) (2008) 47-54.
[22] Y. R. Shih, Y. Hwang, A. Phadke, H. Kang, N. S. Hwang, E. J. Caro, S. Nguyen, M. Siu, E. A. Theodorakis, N.C. Gianneschi, K. S. Vecchio, S. Chien, O. K. Lee, S. Varghese, Calcium phosphate-bearing matrices induce osteogenic differentiation of stem cells through adenosine signaling, Proc Natl Acad Sci USA 111(3) (2014) 990-5.
[23] H. D. Kim, H. L. Jang, H. Y. Aim, H. K. Lee, J. Park, E. S. Lee, E. A. Lee, Y. H. Jeong, D. G. Kim, K. T. Nam, N. S. Hwang, Biomimetic whitlockite inorganic nanoparticles-mediated in situ remodeling and rapid bone regeneration, Biomaterials 112 (2017) 31-43.
[24] K. Jiao, L. N. Niu, Q. H. Li, F. M. Chen, W. Zhao, J. J. Li, J. H. Chen, C. W. Cutler, D. H. Pashley, F. R. Tay, Biphasic silica/apatite co-mineralized collagen scaffolds stimulate osteogenesis and inhibit RANKL-mediated osteoclastogenesis, Acta Biomater 19 (2015) 23-32.
[25] J. C. Lee, C. T. Pereira, X. Ren, W. Huang, D. Bischoff, D. W. Weisgerber, D. T. Yamaguchi, B. A. Harley, T. A. Miller, Optimizing Collagen Scaffolds for Bone Engineering: Effects of Cross-linking and Mineral Content on Structural Contraction and Osteogenesis, J Craniofac Surg (2015).
[26] X. Ren, D. Bischoff, D. W. Weisgerber, M. S. Lewis, V. Tu, D. T. Yamaguchi, T. A. Miller, B. A. Harley, J. C. Lee, Osteogenesis on nanoparticulate mineralized collagen scaffolds via autogenous activation of the canonical BMP receptor signaling pathway, Biomaterials 50 (2015) 107-14.
[27] X. Ren, D. W. Weisgerber, D. Bischoff, M. S. Lewis, R. R. Reid, T. C. He, D. T. Yamaguchi, T. A. Miller, B. A. Harley, J. C. Lee, Nanoparticulate Mineralized Collagen Scaffolds and BMP-9 Induce a Long-Term Bone Cartilage Construct in Human Mesenchymal Stem Cells, Adv Healthc Mater 5(14) (2016) 1821-30.
[28] X. Ren, V. Tu, D. Bischoff, D. W. Weisgerber, M. S. Lewis, D. T. Yamaguchi, T. A. Miller, B. A. Harley, J. C. Lee, Nanoparticulate mineralized collagen scaffolds induce in vivo bone regeneration independent of progenitor cell loading or exogenous growth factor stimulation, Biomaterials 89 (2016) 67-78.
[29] D. W. Weisgerber, S. R. Caliari, B. A. Harley, Mineralized collagen scaffolds induce hMSC osteogenesis and matrix remodeling, Biomater Sci 3(3) (2015) 533-42.
[30] Q. Zhou, X. Ren, D. Bischoff, D. W. Weisgerber, D. T. Yamaguchi, T. A. Miller, B. A. C. Harley, J. C. Lee, Nonmineralized and Mineralized Collagen Scaffolds Induce Differential Osteogenic Signaling Pathways in Human Mesenchymal Stem Cells, Adv Healthc Mater 6(23) (2017).
[31] B. A. Harley, A. K. Lynn, Z. Wissner-Gross, W. Bonfield, I. V. Yannas, L. J. Gibson, Design of a multiphase osteochondral scaffold. II. Fabrication of a mineralized collagen-glycosaminoglycan scaffold, Journal of biomedical materials research. Part A 92(3) (2010) 1066-77.
[32] B. A. Harley, J. H. Leung, E. C. Silva, L. J. Gibson, Mechanical characterization of collagen-glycosaminoglycan scaffolds, Acta biomaterialia 3(4) (2007) 463-74.
[33] D. W. Weisgerber, D. O. Kelkhoff, S. R. Caliari, B. A. Harley, The impact of discrete compartments of a multi-compartment collagen-GAG scaffold on overall construct biophysical properties, Journal of the mechanical behavior of biomedical materials 28 (2013) 26-36.
[34] L. H. Olde Damink, P. J. Dijkstra, M. J. van Luyn, P. B. van Wachem, P. Nieuwenhuis, J. Feijen, Cross-linking of dermal sheep collagen using a water-soluble carbodiimide, Biomaterials 17(8) (1996) 765-73.
[35] S. K. Tat, J. P. Pelletier, D. Lajeunesse, H. Fahmi, N. Duval, J. Martel-Pelletier, Differential modulation of RANKL isoforms by human osteoarthritic subchondral bone osteoblasts: influence of osteotropic factors, Bone 43(2) (2008) 284-91.
[36] T. Ikeda, M. Kasai, J. Suzuki, H. Kuroyama, S. Seki, M. Utsuyama, K. Hirokawa, Multimerization of the receptor activator of nuclear factor-kappaB ligand (RANKL) isoforms and regulation of osteoclastogenesis, J Biol Chem 278(47) (2003) 47217-22.
[37] N. C. Walsh, K. A. Alexander, C. A. Manning, S. Karmakar, S. K. Karmakar, J. F. Wang, C. M. Weyand, A. R. Pettit, E. M. Gravallese, Activated human T cells express alternative mRNA transcripts encoding a secreted form of RANKL, Genes Immun 14(5) (2013) 336-45.
[38] S. Palumbo, W. J. Li, Osteoprotegerin enhances osteogenesis of human mesenchymal stem cells, Tissue Eng Part A 19(19-20) (2013) 2176-87.
[39] H. Yu, P. de Vos, Y. Ren, Overexpression of osteoprotegerin promotes preosteoblast differentiation to mature osteoblasts, Angle Orthod 81(1) (2011) 100-106.
[40] F. Su, S. S. Liu, J. L. Ma, D. S. Wang, L. L. E, H. C. Liu, Enhancement of periodontal tissue regeneration by transplantation of osteoprotegerin-engineered periodontal ligament stem cells, Stem Cell Res Ther 6 (2015) 22.
[41] X. Liu, C. Bao, H. H. Xu, J. Pan, J. Hu, P. Wang, E. Luo, Osteoprotegerin gene-modified BMSCs with hydroxyapatite scaffold for treating critical-sized mandibular defects in ovariectomized osteoporotic rats, Acta Biomater 42 (2016) 378-88.
[42] M. Koide, Y. Kobayashi, T. Yamashita, S. Uehara, M. Nakamura, B. Y. Hiraoka, Y. Ozaki, T. Iimura, H. Yasuda, N. Takahashi, N. Udagawa, Bone Formation Is Coupled to Resorption Via Suppression of Sclerostin Expression by Osteoclasts, J Bone Miner Res 32(10) (2017) 2074-2086.
[43] H. Enomoto, S. Shiojiri, K. Hoshi, T. Furuichi, R. Fukuyama, C. A. Yoshida, N. Kanatani, R. Nakamura, A. Mizuno, A. Zanma, K. Yano, H. Yasuda, K. Higashio, K. Takada, T. Komori, Induction of osteoclast differentiation by Runx2 through receptor activator of nuclear factor-kappa B ligand (RANKL) and osteoprotegerin regulation and partial rescue of osteoclastogenesis in Runx2−/− mice by RANKL transgene, J Biol Chem 278(26) (2003) 23971-7.
[44] Y. H. Gao, T. Shinki, T. Yuasa, H. Kataoka-Enomoto, T. Komori, T. Suda, A. Yamaguchi, Potential role of cbfal, an essential transcriptional factor for osteoblast differentiation, in osteoclastogenesis: regulation of mRNA expression of osteoclast differentiation factor (ODF), Biochem Biophys Res Commun 252(3) (1998) 697-702.
[45] B. Kadriu, P. W. Gold, D. A. Luckenbaugh, M. S. Lener, E. D. Ballard, M. J. Niciu, I.D. Henter, L. T. Park, R. T. De Sousa, P. Yuan, R. Machado-Vieira, C. A. Zarate, Acute ketamine administration corrects abnormal inflammatory bone markers in major depressive disorder, Mol Psychiatry (2017).
[46] F. Gori, L. C. Hofbauer, C. R. Dunstan, T. C. Spelsberg, S. Khosla, B. L. Riggs, The expression of osteoprotegerin and RANK ligand and the support of osteoclast formation by stromal-osteoblast lineage cells is developmentally regulated, Endocrinology 141(12) (2000) 4768-76.
[47] T. Kon, T. J. Cho, T. Aizawa, M. Yamazaki, N. Nooh, D. Graves, L. C. Gerstenfeld, T. A. Einhorn, Expression of osteoprotegerin, receptor activator of NF-kappaB ligand (osteoprotegerin ligand) and related proinflammatory cytokines during fracture healing, J Bone Miner Res 16(6) (2001) 1004-14.
[48] H. Tanaka, T. Mine, H. Ogasa, T. Taguchi, C. T. Liang, Expression of RANKL/OPG during bone remodeling in vivo, Biochem Biophys Res Commun 411(4) (2011) 690-4.
[49] Barradas, A. M., Fernandes, H. A., Groen, N., Chai, Y. C., Schrooten, J., van de Peppel, J., de Boer, J. (2012). A calcium-induced signaling cascade leading to osteogenic differentiation of human bone marrow-derived mesenchymal stromal cells. Biomaterials, 33(11), 3205-3215. doi:10.1016/j.biomaterials.2012.01.020.
[50] Kanatani, M., Sugimoto, T., Kano, J., Kanzawa, M., & Chihara, K. (2003). Effect of high phosphate concentration on osteoclast differentiation as well as bone-resorbing activity. J Cell Physiol, 196(1), 180-189. doi:10.1002/jcp.10270.
[51] Lorget, F., Kamel, S., Mentaverri, R., Wattel, A., Naassila, M., Maamer, M., & Brazier, M. (2000). High extracellular calcium concentrations directly stimulate osteoclast apoptosis. Biochem Biophys Res Commun, 268(3), 899-903. doi:10.1006/bbrc.2000.2229.
[52] Dvorak, M. M., Siddiqua, A., Ward, D. T., Carter, D. H., Dallas, S. L., Nemeth, E. F., & Riccardi, D. (2004). Physiological changes in extracellular calcium concentration directly control osteoblast function in the absence of calciotropic hormones. Proc Natl Acad Sci USA, 101(14), 5140-5145. doi:10.1073/pnas.030614110

SEQUENCE LISTING
Homo sapiens TNF receptor superfamily member llb (TNFRSF11B,
mRNA, osteoprotegerin, OPG), SEQ ID NO 1:
tttttttccc ctgctctccc aggggccaga caccaccgcc ccacccctca cgccccacct
ccctggggga tcctttccgc cccagccctg aaagcgttaa ccctggagct ttctgcacac
cccccgaccg ctcccgccca agcttcctaa aaaagaaagg tgcaaagttt ggtccaggat
agaaaaatga ctgatcaaag gcaggcgata cttcctgttg ccgggacgct atatataacg
tgatgagcgc acgggctgcg gagacgcacc ggagcgctcg cccagccgcc gcctccaagc
ccctgaggtt tccggggacc acaatgaaca acttgctgtg ctgcgcgctc gtgtttctgg
acatctccat taagtggacc acccaggaaa cgtttcctcc aaagtacctt cattatgacg
aagaaacctc tcatcagctg ttgtgtgaca aatgtcctcc tggtacctac ctaaaacaac
actgtacagc aaagtggaag accgtgtgcg ccccttgccc tgaccactac tacacagaca
gctggcacac cagtgacgag tgtctatact gcagccccgt gtgcaaggag ctgcagtacg
tcaagcagga gtgcaatcgc acccacaacc gcgtgtgcga atgcaaggaa gggcgctacc
ttgagataga gttctgcttg aaacatagga gctgccctcc tggatttgga gtggtgcaag
ctggaacccc agagcgaaat acagtttgca aaagatgtcc agatgggttc ttctcaaatg
agacgtcatc taaagcaccc tgtagaaaac acacaaattg cagtgtcttt ggtctcctgc
taactcagaa aggaaatgca acacacgaca acatatgttc cggaaacagt gaatcaactc
aaaaatgtgg aatagatgtt accctgtgtg aggaggcatt cttcaggttt gctgttccta
caaagtttac gcctaactgg cttagtgtct tggtagacaa tttgcctggc accaaagtaa
acgcagagag tgtagagagg ataaaacggc aacacagctc acaagaacag actttccagc
tgctgaagtt atggaaacat caaaacaaag accaagatat agtcaagaag atcatccaag
atattgacct ctgtgaaaac agcgtgcagc ggcacattgg acatgctaac ctcaccttcg
agcagcttcg tagcttgatg gaaagcttac cgggaaagaa agtgggagca gaagacattg
aaaaaacaat aaaggcatgc aaacccagtg accagatcct gaagctgctc agtttgtggc
tggcgaccaa gacaccttga agggcctaat gcacgcacta aagcactcaa agacgtacca
ctttcccaaa actgtcactc agagtctaaa gaagaccatc aggttccttc acagcttcac
aatgtacaaa ttgtatcaga agttattttt agaaatgata ggtaaccagg tccaatcagt
aaaaataagc tgcttataac tggaaatggc cattgagctg tttcctcaca attggcgaga
tcccatggat gagtaaactg tttctcaggc acttgaggct ttcagtgata
tctttctcat taccagtgac taattttgcc acagggtact aaaagaaact atgatgtgga
gaaaggacta acatctcctc caataaaccc caaatggtta atccaactgt cagatctgga
tcgttatcta ctgactatat tttcccttat tactgcttgc agtaattcaa ctggaaatta
aaaaaaaaaa actagactcc attgtgcctt actaaatatg ggaatgtcta acttaaatag
ctttgagatt tcagctatgc tagaggcttt tattagaaag ccatattttt ttctgtaaaa
gttactaata tatctgtaac actattacag tattgctatt tatattcatt cagatataag
atttgtacat attatcatcc tataaagaaa cggtatgact taattttaga aagaaaatta
tattctgttt attatgacaa atgaaagaga aaatatatat ttttaatgga aagtttgtag
catttttcta ataggtactg ccatattttt ctgtgtggag tatttttata attttatctg
tataagctgt aatatcattt tatagaaaat gcattattta gtcaattgtt taatgttgga
aaacatatga aatataaatt atctgaatat tagatgctct gagaaattga atgtacctta
tttaaaagat tttatggttt tataactata taaatgacat tattaaagtt ttcaaattat
tttttaaaaa aaaa
Homo sapiens TNF receptor superfamily member llb (TNFRSF11B,
osteoprotegerin, OPG) protein sequence, SEQ ID NO: 2:
msaraaethr sarpaaaskp lrfpgttmnk llccalvfld isikwttqet fppkylhyde
etshqllcdk cppgtylkqh ctakwktvca pcpdhyytds whtsdeclyc spvckelqyv
kqecnrthnr vceckegryl eiefclkhrs cppgfgvvqa gtperntvck rcpdgffsne
tsskaperkh tncsvfg111 tqkgnathdn icsgnsestq kcgidvtice eaffrfavpt
kftpnwlsvl vdnlpgtkvn aesverikrq hssqeqtfql lklwkhqnkd qdivkkiiqd
idlcensvqr highanitfe qlrslmeslp gkkvgaedie ktikackpsd qilkllslwr
ikngdqdtlk glmhalkhsk tyhfpktvtq slkktirflh sftmyklyqk lflemignqv
qsvkiscl

1. A composition comprising a collagen glycosaminoglycan scaffold and one or more of an osteoprotegerin (OPG), an OPG fragment or an equivalent of each thereof.

2. The composition of claim 1, wherein the collagen glycosaminoglycan scaffold is a nanoparticulate mineralized collagen glycosaminoglycan (MC-GAG) scaffold.

3. (canceled)

4. The composition of claim 1, wherein the OPG, the OPG fragment or an equivalent of each thereof;
is provided by a mesenchymal stem cell (MSC) or a cell differentiated from a MSC, that expresses the (OPG), the OPG fragment or the equivalent of each thereof; or
is expressed at a level above endogenously expressed OPG; or
is expressed at about 5 ng/mL to about 20 ng/mL; or
is recombinant; or
is encoded by a nucleic acid, wherein the nucleic acid comprises:
(i) a polynucleotide of SEQ ID NO: 1 or a polynucleotide that encodes SEQ ID NO: 2;
(ii) a polynucleotide comprising a biological equivalent of SEQ ID NO: 1 or a polynucleotide that encodes SEQ ID NO: 2;
(iii) a polynucleotide having at least 80% sequence identity to SEQ ID NO: 1 or a polynucleotide that encodes SEQ ID NO: 2; or
(iv) a fragment of the polynucleotide of any one of (i)-(iii) that encodes functional OPG.

5.-8. (canceled)

9. The composition of claim 4, wherein the nucleic acid is operatively linked to one or more regulatory elements that provide for expression of the nucleic acid, optionally wherein the nucleic acid and the one or more regulatory elements are comprised within a vector.

10. The composition of claim 9, wherein the vector is a eukaryotic vector or a prokaryotic vector.

11. The composition of claim 10, wherein the eukaryotic vector is selected from the group of: an adenoviral vector an alphaviral vector, an adeno-associated viral vector (AAV), and a lentiviral vector.

12. The composition of claim 4, wherein the MSC:
is a bone marrow derived MSC;
is selected from the group of: a bovine MSC, a feline MSC, a canine MSC, a murine MSC, an equine MSC, and a human MSC; or
has a cell marker profile comprising: CD105⁺, CD166⁺, CD29⁺, CD44⁺, CD14-, CD34, and CD45.

13.-15. (canceled)

16. The composition of claim 4, wherein the cell differentiated from a MSC is an osteoblast or an osteocyte.

17. The composition of claim 1, further comprising a carrier.

18. (canceled)

19. A method of promoting osteogenesis in a subject in need thereof, comprising:
administering to the subject an effective amount of the composition of claim 1.

20. A method of attenuating bone resorption in a subject in need thereof, comprising:
administering to the subject an effective amount of the composition of claim 1.

21. A method of inhibiting osteoclastogenesis in a subject in need thereof, comprising:
administering to the subject an effective amount of the composition of claim 1.

22. A method of inhibiting osteoclast activation in a subject in need thereof, comprising:
administering to the subject an effective amount of the composition of claim 1.

23.-27. (canceled)

28. A method of preparing a composition, comprising contacting a MC-GAG scaffold with a solution comprising OPG, an OPG fragment or an equivalent of each thereof.

29. (canceled)

30. A method of preparing a composition comprising:
culturing in a differentiation medium a MSC expressing exogenous osteoprotegerin (OPG), an exogenous OPG fragment or an equivalent of each thereof; within a collagen glycosaminoglycan scaffold.

31. The method of claim 30, wherein the differentiation medium comprises one or more of a compound selected from the group of b-glycerophosphate, ascorbic acid, and dexamethasone.

32. The method of claim 30, wherein the composition comprises a mesenchymal stem cell (MSC) expressing exogenous osteoprotegerin (OPG), an exogenous OPG, a fragment or an equivalent of each thereof and wherein the exogenous osteoprotegerin (OPG), the exogenous OPG fragment or the equivalent of each thereof is recombinant.

33. The method of claim 30, wherein the composition comprises a mesenchymal stem cell (MSC) expressing exogenous osteoprotegerin (OPG), an exogenous OPG fragment or an equivalent of each thereof, and wherein the MSC has been transduced with a virus comprising a nucleic acid encoding OPG, an OPG fragment or an equivalent of each thereof.

34. The method of claim 33, wherein the nucleic acid encoding OPG, an OPG fragment or an equivalent of each thereof comprises the polynucleotide of SEQ ID NO: 1, a biological equivalent thereof, or a nucleic acid having at least 80% sequence identity to SEQ ID NO: 1 or a polynucleotide encoding the amino acid sequence of SEQ ID NO: 2, a biological equivalent thereof, or a nucleic acid having at least 80% sequence identity to the polynucleotide encoding SEQ ID NO: 2.

35.-43. (canceled)

44. A kit comprising a composition of claim 1, and instructions for use.


SEQUENCE LISTING (10 sequences)

SEQ ID NO: 1 - DNA, 2344 bp, Homo sapiens
tttttttcccctgctctcccaggggccagacaccaccgccccacccctcacgccccacctccctgggggatcctttccgccccagccctgaaagcgttaaccctggagctttctgcacaccccccgaccgctcccgcccaagcttcctaaaaaagaaaggtgcaaagtttggtccaggatagaaaaatgactgatcaaaggcaggcgatacttcctgttgccgggacgctatatataacgtgatgagcgcacgggctgcggagacgcaccggagcgctcgcccagccgccgcctccaagcccctgaggtttccggggaccacaatgaacaacttgctgtgctgcgcgctcgtgtttctggacatctccattaagtggaccacccaggaaacgtttcctccaaagtaccttcattatgacgaagaaacctctcatcagctgttgtgtgacaaatgtcctcctggtacctacctaaaacaacactgtacagcaaagtggaagaccgtgtgcgccccttgccctgaccactactacacagacagctggcacaccagtgacgagtgtctatactgcagccccgtgtgcaaggagctgcagtacgtcaagcaggagtgcaatcgcacccacaaccgcgtgtgcgaatgcaaggaagggcgctaccttgagatagagttctgcttgaaacataggagctgccctcctggatttggagtggtgcaagctggaaccccagagcgaaatacagtttgcaaaagatgtccagatgggttcttctcaaatgagacgtcatctaaagcaccctgtagaaaacacacaaattgcagtgtctttggtctcctgctaactcagaaaggaaatgcaacacacgacaacatatgttccggaaacagtgaatcaactcaaaaatgtggaatagatgttaccctgtgtgaggaggcattcttcaggtttgctgttcctacaaagtttacgcctaactggcttagtgtcttggtagacaatttgcctggcaccaaagtaaacgcagagagtgtagagaggataaaacggcaacacagctcacaagaacagactttccagctgctgaagttatggaaacatcaaaacaaagaccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggctggcgaccaagacaccttgaagggcctaatgcacgcactaaagcactcaaagacgtaccactttcccaaaactgtcactcagagtctaaagaagaccatcaggttccttcacagcttcacaatgtacaaattgtatcagaagttatttttagaaatgataggtaaccaggtccaatcagtaaaaataagctgcttataactggaaatggccattgagctgtttcctcacaattggcgagatcccatggatgagtaaactgtttctcaggcacttgaggctttcagtgatatctttctcattaccagtgactaattttgccacagggtactaaaagaaactatgatgtggagaaaggactaacatctcctccaataaaccccaaatggttaatccaactgtcagatctggatcgttatctactgactatattttcccttattactgcttgcagtaattcaactggaaattaaaaaaaaaaaactagactccattgtgccttactaaatatgggaatgtctaacttaaatagctttgagatttcagctatgctagaggcttttattagaaagccatatttttttctgtaaaagttactaatatatctgtaacactattacagtattgctatttatattcattcagatataagatttgtacatattatcatcctataaagaaacggtatgacttaattttagaaagaaaattatattctgtttattatgacaaatgaaagagaaaatatatatttttaatggaaagtttgtagcatttttctaataggtactgccatatttttctgtgtggagtatttttataattttatctgtataagctgtaatatcattttatagaaaatgcattatttagtcaattgtttaatgttggaaaacatatgaaatataaattatctgaatattagatgctctgagaaattgaatgtaccttatttaaaagattttatggttttataactatataaatgacattattaaagttttcaaattattttttaaaaaaaaa

SEQ ID NO: 2 - protein, 428 aa, Homo sapiens
MSARAAETHRSARPAAASKPLRFPGTTMNKLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 3 - DNA, 22 bp, Artificial Sequence
  Description of Artificial Sequence Synthetic primer
tagagtgttcaaagcaggcccg

SEQ ID NO: 4 - DNA, 22 bp, Artificial Sequence
  Description of Artificial Sequence Synthetic primer
tccctcttaatcatggcctcag

SEQ ID NO: 5 - DNA, 22 bp, Artificial Sequence
  Description of Artificial Sequence Synthetic primer
aagccggtgcctgggtggccat

SEQ ID NO: 6 - DNA, 20 bp, Artificial Sequence
  Description of Artificial Sequence Synthetic primer
acaggagagtcgcttcagag

SEQ ID NO: 7 - DNA, 20 bp, Artificial Sequence
  Description of Artificial Sequence Synthetic primer
tcggagaggtaccagatggg

SEQ ID NO: 8 - DNA, 20 bp, Artificial Sequence
  Description of Artificial Sequence Synthetic primer
aactcttgcctcgtccactc

SEQ ID NO: 9 - DNA, 20 bp, Artificial Sequence
  Description of Artificial Sequence Synthetic primer
ggactgccagaggaagcaat

SEQ ID NO: 10 - DNA, 21 bp, Artificial Sequence
  Description of Artificial Sequence Synthetic primer
ggcctgtacttaaagacccca